SEQUENCE LISTING (24 sequences)

SEQ ID NO: 1 - protein, 8 aa, Artificial Sequence
  Polypeptide sequence
GGSIRNNY

SEQ ID NO: 2 - protein, 7 aa, Artificial Sequence
  Polypeptide sequence
IYISGTT

SEQ ID NO: 3 - protein, 13 aa, Artificial Sequence
  Polypeptide sequence
AREHYVSGTSLDN

SEQ ID NO: 4 - protein, 7 aa, Artificial Sequence
  Polypeptide sequence
QSVNTNY

SEQ ID NO: 5 - protein, 3 aa, Artificial Sequence
  Polypeptide sequence
GTS

SEQ ID NO: 6 - protein, 9 aa, Artificial Sequence
  Polypeptide sequence
QQDYNLPYT

SEQ ID NO: 7 - protein, 8 aa, Artificial Sequence
  Polypeptide sequence
GFTFSSYG

SEQ ID NO: 8 - protein, 8 aa, Artificial Sequence
  Polypeptide sequence
FWYDGSNK

SEQ ID NO: 9 - protein, 12 aa, Artificial Sequence
  Polypeptide sequence
ARAPNWDDAFDI

SEQ ID NO: 10 - protein, 9 aa, Artificial Sequence
  Polypeptide sequence
SGSVSTSYY

SEQ ID NO: 11 - protein, 3 aa, Artificial Sequence
  Polypeptide sequence
STN

SEQ ID NO: 12 - protein, 10 aa, Artificial Sequence
  Polypeptide sequence
VLFMGSGIWV

SEQ ID NO: 13 - protein, 119 aa, Artificial Sequence
  Polypeptide sequence
QEQLQESGPGLVKPSETLSLTCTVSGGSIRNNYYNWIRQPAGKGLEWIGRIYISGTTNSNPSLKSRVTMSIDTSKNQFSLKLSSVTAADTAIYYCAREHYVSGTSLDNWGQGTLVTVSS

SEQ ID NO: 14 - protein, 108 aa, Artificial Sequence
  Polypeptide sequence
EIVMTQSPTTLSLSPGERATLSCRASQSVNTNYFSWYQQKPGLTPRLLIYGTSTRATGIPARFSGSGSGTDFTLTISSLQPEDFGIYYCQQDYNLPYTFGQGTYLEIK

SEQ ID NO: 15 - protein, 119 aa, Artificial Sequence
  Polypeptide sequence
QVQLVESGGGVVQPGRSLRLSCAASGFTFSSYGMHWVRQAPGKGLEWVAVFWYDGSNKYYADSVKGRFTISRDNSKNTLYLQMNSLSAEDTAVYYCARAPNWDDAFDIWGQGTMVTVSS

SEQ ID NO: 16 - protein, 110 aa, Artificial Sequence
  Polypeptide sequence
QTVVTQEPSFSVSPGGTVTLTCGLSSGSVSTSYYPNWYQQTPGQAPRTLIYSTNTRSSGVPDRFSGSILGNKAALTITGAQADDESDYYCVLFMGSGIWVFGGGTKLTVL

SEQ ID NO: 17 - protein, 448 aa, Artificial Sequence
  Polypeptide sequence
QEQLQESGPGLVKPSETLSLTCTVSGGSIRNNYYNWIRQPAGKGLEWIGRIYISGTTNSNPSLKSRVTMSIDTSKNQFSLKLSSVTAADTAIYYCAREHYVSGTSLDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSRLTVDKSRWQQGNVFSCSVMHEALHNHYTQKSLSLSPG

SEQ ID NO: 18 - protein, 448 aa, Artificial Sequence
  Polypeptide sequence
QEQLQESGPGLVKPSETLSLTCTVSGGSIRNNYYNWIRQPAGKGLEWIGRIYISGTTNSNPSLKSRVTMSIDTSKNQFSLKLSSVTAADTAIYYCAREHYVSGTSLDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVMHEALHNHYTQKSLSLSPG

SEQ ID NO: 19 - protein, 215 aa, Artificial Sequence
  Polypeptide sequence
EIVMTQSPTTLSLSPGERATLSCRASQSVNTNYFSWYQQKPGLTPRLLIYGTSTRATGIPARFSGSGSGTDFTLTISSLQPEDFGIYYCQQDYNLPYTFGQGTYLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 448 aa, Artificial Sequence
  Polypeptide sequence
QVQLVESGGGVVQPGRSLRLSCAASGFTFSSYGMHWVRQAPGKGLEWVAVFWYDGSNKYYADSVKGRFTISRDNSKNTLYLQMNSLSAEDTAVYYCARAPNWDDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSRLTVDKSRWQQGNVFSCSVMHEALHNHYTQKSLSLSPG

SEQ ID NO: 21 - protein, 448 aa, Artificial Sequence
  Polypeptide sequence
QVQLVESGGGVVQPGRSLRLSCAASGFTFSSYGMHWVRQAPGKGLEWVAVFWYDGSNKYYADSVKGRFTISRDNSKNTLYLQMNSLSAEDTAVYYCARAPNWDDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVMHEALHNHYTQKSLSLSPG

SEQ ID NO: 22 - protein, 216 aa, Artificial Sequence
  Polypeptide sequence
QTVVTQEPSFSVSPGGTVTLTCGLSSGSVSTSYYPNWYQQTPGQAPRTLIYSTNTRSSGVPDRFSGSILGNKAALTITGAQADDESDYYCVLFMGSGIWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 23 - protein, 523 aa, Artificial Sequence
  Polypeptide sequence
WELTILHTNDVHSRLEQTSEDSSKCVNASRCMGGVARLFTKVQQIRRAEPNVLLLDAGDQYQGTIWFTVYKGAEVAHFMNALRYDAMALGNHEFDNGVEGLIEPLLKEAKFPILSANIKAKGPLASQISGLYLPYKVLPVGDEVVGIVGYTSKETPFLSNPGTNLVFEDEITALQPEVDKLKTLNVNKIIALGHSGFEMDKLIAQKVRGVDVVVGGHSNTFLYTGNPPSKEVPAGKYPFIVTSDDGRKVPVVQAYAFGKYLGYLKIEFDERGNVISSHGNPILLNSSIPEDPSIKADINKWRIKLDNYSTQELGKTIVYLDGSSQSCRFRECNMGNLICDAMINNNLRHTDEMFWNHVSMCILNGGGIRSPIDERNNGTITWENLAAVLPFGGTFDLVQLKGSTLKKAFEHSVHRYGQSTGEFLQVGGIHVVYDLSRKPGDRVVKLDVLCTKCRVPSYDPLKMDEVYKVILPNFLANGGDGFQMIKDELLRHDSGDQDINVVSTYISKMKVIYPAVEGRIKFS

SEQ ID NO: 24 - protein, 523 aa, Artificial Sequence; identical to SEQ ID NO: 23
  Polypeptide sequence

(19) United States
(12) Patent Application Publication
(10) Pub. No.: US 2022/0403041 A1
(43) Pub. Date: Dec. 22, 2022

(54) BIPARATOPIC CD73 ANTIBODIES

(71) Applicant: GENZYME CORPORATION, Cambridge, MA (US)

(72) Inventors: Dana LORD, Cambridge, MA (US); Katarina RADOSEVIC, Rosny Sous Bois (FR); James E. STEFANO, Cambridge, MA (US); Yenfang ZHOU, Cambridge, MA (US)

(21) Appl. No.: 17/775,446

(22) PCT Filed: Nov. 13, 2020
(86) PCT No.: PCT/US2020/060434

§ 371 Date: May 9, 2022

Related U.S. Application Data
(60) Provisional application No. 63/086,982, filed on Oct. 2, 2020.
(60) Provisional application No. 63/023,542, filed on May 12, 2020.
(60) Provisional application No. 62/936,119, filed on Nov. 15, 2019.

(51) Int. Cl. C07K 16/28; A61P 35/00

(52) U.S. Cl. CPC C07K 16/2896; A61P 35/00; C07K 2317/565; C07K 2317/24; C07K 2317/52

(57) ABSTRACT

Anti-CD73 antigen-binding proteins are provided. Biparatopic anti-CD73 antigen-binding proteins are provided. Methods of inhibiting CD73 activity and methods of treating CD73-mediated diseases and disorders are also provided.

### RELATED APPLICATIONS

The instant application claims priority to U.S. provisional application No. 62/936,119, filed Nov. 15, 2019, U.S. provisional application No. 63/023,542, filed May 12, 2020, and U.S. provisional application No. 63/086,982, filed Oct. 2, 2020, the contents of each application are incorporated herein by reference for all purposes.

### SEQUENCE LISTING

The instant application contains a Sequence Listing which has been submitted electronically in ASCII format and is hereby incorporated by reference in its entirety. Said ASCII copy, created on Nov. 5, 2020, is named 711174_SA9-282PC_ST25.txt and is 35,615 bytes in size.

### FIELD OF THE INVENTION

This disclosure relates to compositions and methods of making bispecific antigen-binding proteins.

### BACKGROUND

CD73 (ecto-5′-nucleotidase, NTSE) is a glycosylated 125 kDa homodimeric membrane bound enzyme which dephosphorylates adenosine monophosphate (AMP) in the extracellular milieu to adenosine (ADO) (Allard et al. 2016. Immunotherapy 8:145-163). Adenosine has potent immunosuppressive effects in the tumor microenvironment so CD73 has attracted wide interest as a target for cancer therapy (Allard et al. 2017. Immunological reviews 276:121-144; Allard et al. 2019. Immunology letters 205:31-39; Kats et al. 2018. International journal of molecular sciences 156:451-457; Sek et al. 2018. Int J Mol Sci. 19(12) pii: E3837; Yang et al. 2018. Current medicinal chemistry. 25:2260-2271). CD73 expression is associated with resistance to anti-HER2 therapy (Turcotte et al. 2017. Cancer research. 77:5652-5663), poor prognosis with reduced anti-tumor immune response in a variety of tumor types (Allard 2016, supra) and the increased growth of tumor cells, migration and invasion in vitro (Zhi et al. 2007. Clinical & experimental metastasis. 24:439-448). A number of clinical studies are in progress with CD73-specific antibodies (Siu et al. 2018. Cancer research. 78:CT180-CT180) and small molecule inhibitors (Overman et al. 2018. Journal of Clinical Oncology. 36(15):4123), alone or in combination with A2a adenosine receptor antagonists and antibodies to other targets, particularly the PD-1/PD-L1 axis (Leone et al. 2018. Journal for immunotherapy of cancer. 6:57). MEDI9447 (oleclumab), a CD73 specific internalizing antibody with moderate inhibition of enzymatic activity, has shown some clinical efficacy as a monotherapy and in combination with the PD-L1 blocker durvalumab (Hay et al. 2016. Oncoimmunology. 5: e1208875). Nevertheless, there is a need in the art for CD73 antibodies with greater clinical efficacy as a monotherapy and in combination with other therapeutics.

There are also indications that CD73 antibodies can exert effects independent of adenosine production. One study indicated that the enhancement of the immune response was mediated through FcγRIV-engagement in mice (Vijayan et al. 2017. Oncoimmunology. 6(5):e1312044) and other work suggested a role for CD73 internalization at suppressing metastasis (Overman 2018, supra; Hay 2016, supra; Terp et al. 2013. Journal of immunology. 191:4165-4173). Nonetheless, adenosine levels in tumors can reach micromolar concentrations, so incomplete inhibition of CD73 activity may be a limiting factor for the efficacy of current CD73-tageting therapeutics (Blay et al. 1997. Cancer research. 57:2602-2605). Thus, the mechanism by which CD73 affects cancer progression may be complex, suggesting the need for very potent inhibition of enzymatic activity or a combination of mechanisms to achieve optimal efficacy.

Achieving potent inhibition of CD73 enzymatic activity (e.g., both a high percentage inhibition and a low EC50) by conventional monospecific CD73 antibodies can be challenging (Geoghegan et al. 2016. mAbs. 8:454-467; WO2016055609A1; WO2017118613). Accordingly, there is a need in the art to identify anti-CD73 antibodies that achieve effective inhibition of CD73 enzymatic activity. Such anti-CD73 antibodies may be useful in the treatment of CD73-mediated diseases and disorders.

### SUMMARY

Disclosed herein are bi-paratopic binding proteins that bind to two different epitopes on CD73 and are capable of achieving potent inhibition of CD73 activity. The binding proteins of the invention are particularly suitable for treating CD73-mediated diseases and disorders.

In one aspect, the disclosure provides an antigen-binding protein or fragment thereof with binding specificity to a CD73 epitope, comprising: (a) an antibody heavy chain variable (VH) domain comprising a CDR-H1 sequence comprising the amino acid sequence of GGSIRNNY (SEQ ID NO: 1) or GFTFSSYG (SEQ ID NO: 7), a CDR-H2 sequence comprising the amino acid sequence of IYISGTT (SEQ ID NO: 2) or FWYDGSNK (SEQ ID NO: 8), and a CDR-H3 sequence comprising the amino acid sequence of AREHYVSGTSLDN (SEQ ID NO: 3) or ARAPNWDDAFDI (SEQ ID NO: 9); and (b) an antibody light chain variable (VL) domain comprising a CDR-L1 sequence comprising the amino acid sequence of QSVNTNY (SEQ ID NO: 4) or SGSVSTSYY (SEQ ID NO: 10), a CDR-L2 sequence comprising the amino acid sequence of GTS (SEQ ID NO: 5) or STN (SEQ ID NO: 11), and a CDR-L3 sequence comprising the amino acid sequence of QQDYNLPYT (SEQ ID NO: 6) or VLFMGSGIWV (SEQ ID NO: 12).

In certain embodiments, the VH domain comprises the amino acid sequence of SEQ ID NO: 13 or SEQ ID NO: 15, and the VL domain comprises the amino acid sequence of SEQ ID NO: 14 or SEQ ID NO: 16.

In certain embodiments, the antibody heavy chain comprises the amino acid sequence of SEQ ID NO: 17, 18, 20, or 21, and the antibody light chain comprises the amino acid sequence of SEQ ID NO: 19 or SEQ ID NO: 22.

In certain embodiments, the antigen binding protein or fragment thereof comprises a VH domain at least about 90% identical or at least about 95% identical to the amino acid sequence of SEQ ID NO: 13 or SEQ ID NO: 15, and a VL domain at least about 90% identical or at least about 95% identical to the amino acid sequence of SEQ ID NO: 14 or SEQ ID NO: 16.

In certain embodiments, the antigen binding protein or fragment thereof comprises an antibody heavy chain at least about 90% identical or at least about 95% identical to the amino acid sequence of SEQ ID NO: 17, 18, 20, or 21, and an antibody light chain at least about 90% identical or at least about 95% identical to the amino acid sequence of SEQ ID NO: 19 or SEQ ID NO: 22.

In certain embodiments, the antigen binding protein or fragment thereof comprises: (a) the VH domain comprises a CDR-H1 sequence comprising the amino acid sequence of GGSIRNNY (SEQ ID NO: 1), a CDR-H2 sequence comprising the amino acid sequence of IYISGTT (SEQ ID NO: 2), and a CDR-H3 sequence comprising the amino acid sequence of AREHYVSGTSLDN (SEQ ID NO: 3); and (b) the VL domain comprises a CDR-L1 sequence comprising the amino acid sequence of QSVNTNY (SEQ ID NO: 4), a CDR-L2 sequence comprising the amino acid sequence of GTS (SEQ ID NO: 5), and a CDR-L3 sequence comprising the amino acid sequence of QQDYNLPYT (SEQ ID NO: 6).

7 In certain embodiments, the VH domain comprises the amino acid sequence of SEQ ID NO: 13, and the VL domain comprises the amino acid sequence of SEQ ID NO: 14.

In certain embodiments, the antibody heavy chain comprises the amino acid sequence of SEQ ID NO: 17 or SEQ ID NO: 18, and the antibody light chain comprises the amino acid sequence of SEQ ID NO: 19.

In certain embodiments, the antigen binding protein or fragment thereof comprises: (a) the VH domain comprises a CDR-H1 sequence comprising the amino acid sequence of GFTFSSYG (SEQ ID NO: 7), a CDR-H2 sequence comprising the amino acid sequence of FWYDGSNK (SEQ ID NO: 8), and a CDR-H3 sequence comprising the amino acid sequence of ARAPNWDDAFDI (SEQ ID NO: 9); and (b) the VL domain comprises a CDR-L1 sequence comprising the amino acid sequence of SGSVSTSYY (SEQ ID NO: 10), a CDR-L2 sequence comprising the amino acid sequence of STN (SEQ ID NO: 11), and a CDR-L3 sequence comprising the amino acid sequence of VLFMGSGIWV (SEQ ID NO: 12).

In certain embodiments, the VH domain comprises the amino acid sequence of SEQ ID NO: 15, and the VL domain comprises the amino acid sequence of SEQ ID NO: 16.

In certain embodiments, the antibody heavy chain comprises the amino acid sequence of SEQ ID NO: 20 or SEQ ID NO: 21, and the antibody light chain comprises the amino acid sequence of SEQ ID NO: 22.

In certain embodiments, the antigen binding protein binds a human CD73 polypeptide comprising the amino acid sequence of SEQ ID NO: 23.

In certain embodiments, the antigen binding protein binds an epitope of human CD73 polypeptide comprising the amino acids N96, G97, V98, E99, K121, P123, P156, F157, S159, N160, G162, T163, N164, L165, V166, F167, E168, R491, and D496 of SEQ ID NO: 23.

In certain embodiments, the antigen binding protein binds an epitope of human CD73 polypeptide comprising the amino acids P112, K119, A125, S126, S129, G130, L133, P134, Y135, K136, K180, L184, and N185 of SEQ ID NO: 23.

In certain embodiments, the antigen binding protein is a chimeric or humanized antibody. In certain embodiments, the antigen binding protein is a human antibody.

In certain embodiments, the antigen binding protein is a monoclonal antibody.

In certain embodiments, the antigen binding protein comprises one or more full-length antibody heavy chains comprising an Fc region. In certain embodiments, the Fc region is a human IgG1 Fc region.

In certain embodiments, the human IgG1 Fc region comprises amino acid substitutions at one or more positions corresponding to positions 405 and 409 of human IgG1 according to EU Index, wherein the amino acid substitutions are F405L and K409R.

In one aspect, the disclosure provides a pharmaceutical composition comprising the antigen binding protein or fragment thereof recited above, and a pharmaceutically acceptable carrier.

In one aspect, the disclosure provides an isolated nucleic acid molecule encoding the antigen binding protein or fragment thereof of recited above.

In one aspect, the disclosure provides an expression vector comprising the nucleic acid molecule recited above.

In one aspect, the disclosure provides a host cell comprising the expression vector recited above.

In one aspect, the disclosure provides a biparatopic antigen-binding protein comprising binding specificity to a first CD73 epitope and a second CD73 epitope.

In certain embodiments, the biparatopic antigen-binding protein comprises: (a) a first VH domain with specificity to the first CD73 epitope comprising a CDR-H1 sequence comprising the amino acid sequence of GGSIRNNY (SEQ ID NO: 1), a CDR-H2 sequence comprising the amino acid sequence of IYISGTT (SEQ ID NO: 2), and a CDR-H3 sequence comprising the amino acid sequence of AREHYVSGTSLDN (SEQ ID NO: 3); (b) a first VL domain with specificity to the first CD73 epitope comprising a CDR-L1 sequence comprising the amino acid sequence of QSVNTNY (SEQ ID NO: 4), a CDR-L2 sequence comprising the amino acid sequence of GTS (SEQ ID NO: 5), and a CDR-L3 sequence comprising the amino acid sequence of QQDYNLPYT (SEQ ID NO: 6); (c) a second VH domain with specificity to the second CD73 epitope comprises a CDR-H1 sequence comprising the amino acid sequence of GFTFSSYG (SEQ ID NO: 7), a CDR-H2 sequence comprising the amino acid sequence of FWYDGSNK (SEQ ID NO: 8), and a CDR-H3 sequence comprising the amino acid sequence of ARAPNWDDAFDI (SEQ ID NO: 9); and (d) a second VL domain with specificity to the second CD73 epitope comprises a CDR-L1 sequence comprising the amino acid sequence of SGSVSTSYY (SEQ ID NO: 10), a CDR-L2 sequence comprising the amino acid sequence of STN (SEQ ID NO: 11), and a CDR-L3 sequence comprising the amino acid sequence of VLFMGSGIWV (SEQ ID NO: 12).

In certain embodiments, the first VH domain comprises the amino acid sequence of SEQ ID NO: 13; the second VH domain comprises the amino acid sequence of SEQ ID NO: 15; the first VL domain comprises the amino acid sequence of SEQ ID NO: 14; and the second VL domain comprises the amino acid sequence of SEQ ID NO: 16.

In certain embodiments, the biparatopic antigen-binding protein comprises: (a) a first antibody heavy chain comprises the amino acid sequence of SEQ ID NO: 17 or SEQ ID NO: 18; (b) a second antibody heavy chain comprises the amino acid sequence of SEQ ID NO: 20 or SEQ ID NO: 21; (c) a first antibody light chain comprises the amino acid sequence of SEQ ID NO: 19; and (d) a second antibody light chain comprises the amino acid sequence of SEQ ID NO: 22.

In certain embodiments, the biparatopic antigen-binding protein comprises: (a) a first antibody heavy chain comprises the amino acid sequence of SEQ ID NO: 17; (b) a second antibody heavy chain comprises the amino acid sequence of SEQ ID NO: 21; (c) a first antibody light chain comprises the amino acid sequence of SEQ ID NO: 19; and (d) a second antibody light chain comprises the amino acid sequence of SEQ ID NO: 22.

In certain embodiments, the biparatopic antigen-binding protein comprises: (a) a first antibody heavy chain comprises the amino acid sequence of SEQ ID NO: 18; (b) a second antibody heavy chain comprises the amino acid sequence of SEQ ID NO: 20; (c) a first antibody light chain comprises the amino acid sequence of SEQ ID NO: 19; and (d) a second antibody light chain comprises the amino acid sequence of SEQ ID NO: 22.

In certain embodiments, the biparatopic antigen-binding protein comprises: (a) the first VH and VL domains bind a first epitope of human CD73 polypeptide comprising the amino acids N96, G97, V98, E99, K121, P123, P156, F157, S159, N160, G162, T163, N164, L165, V166, F167, E168, R491, and D496 of SEQ ID NO: 23; and (b) the second VH and VL domains bind a second epitope of human CD73 polypeptide comprising the amino acids P112, K119, A125, S126, S129, G130, L133, P134, Y135, K136, K180, L184, and N185 of SEQ ID NO: 23.

In certain embodiments, the biparatopic antigen-binding protein comprises higher inhibitory activity of CD73 compared to one or both of the monospecific parental antibodies.

In certain embodiments, the biparatopic antigen-binding protein comprises higher inhibitory activity of CD73 compared to the combination of monospecific parental antibodies.

In certain embodiments, the first VH and VL domains bind a first CD73 epitope on a first CD73 dimer molecule, and the second VH and VL domains bind a second CD73 epitope on a second CD73 dimer molecule.

In certain embodiments, the antigen-binding protein is capable of crosslinking two or more CD73 dimer molecules.

In certain embodiments, the biparatopic antigen-binding protein is produced by Fab arm exchange.

In certain embodiments, the Fab arm exchange is performed following the steps of: (a) mixing a first parental, monospecific antigen-binding protein comprising an IgG1 Fc region comprising an amino acid substitution F405L according to EU Index, and a second parental, monospecific antigen-binding protein comprising an IgG1 Fc region comprising an amino acid substitution K409R according to EU Index, to produce a mixture; (b) placing the mixture of step (a) under reducing conditions to produce a reduced antigen-binding protein mixture containing the biparatopic, bispecific antigen-binding protein; (c) placing the mixture of step (b) under oxidizing conditions to reform the disulfide linkages between the heavy chains of the biparatopic, bispecific antigen-binding protein; and (d) isolating the biparatopic, bispecific antigen-binding protein.

In certain embodiments, the first parental, monospecific antigen-binding protein and second parental, monospecific antigen-binding protein are mixed at equimolar amounts.

In certain embodiments, the reducing conditions are produced by adding a reducing agent. In certain embodiments, the reducing agent comprises mercaptoethylamine (MEA).

In certain embodiments, the mixture of step (a) is placed under reducing conditions for about 3 hours to about 6 hours at a temperature of about 18° C. to about 30° C.

In another aspect, the disclosure provides a method for treating a CD73-mediated disease or disorder in a subject, comprising administering to a subject in need thereof the antigen binding protein or fragment thereof of recited above.

In certain embodiments, the CD73-mediated disease or disorder is cancer.

In another aspect, the disclosure provides a method of selecting biparatopic antigen-binding proteins comprising higher inhibitory activity of CD73 compared to one or more monospecific parental antibodies, the method comprising the steps of: a) combining two parental antibodies under conditions that form a biparatopic antigen-binding protein; b) testing the biparatopic antigen-binding protein and one or both of the two parental antibodies in a CD73 activity assay; c) comparing the CD73 activity with the biparatopic antigen-binding protein to the CD73 activity with one or both of the two parental antibodies; and d) selecting the biparatopic antigen-binding protein if the CD73 activity is less than the CD73 activity of one or both of the two parental antibodies.

In certain embodiments, the CD73 activity assay measures adenosine formation. In certain embodiments, the adenosine formation is quantitated by liquid chromatography-mass spectrometry (LC/MS).

In certain embodiments, the CD73 activity assay is performed with COR-L23 lung carcinoma cells expressing human CD73.

### BRIEF DESCRIPTION OF THE DRAWINGS

The foregoing and other features and advantages of the present invention will be more fully understood from the following detailed description of illustrative embodiments taken in conjunction with the accompanying drawings. The patent or application file contains at least one drawing executed in color. Copies of this patent or patent application publication with color drawing(s) will be provided by the Office upon request and payment of the necessary fee.

FIG. 1 depicts a screen of inhibitory activity against CD73 on COR-L23 cells. The inhibition of CD73 activity (%) was determined following exposure to antibodies for 4 hours using a LC/MS based assay with a heavy-isotope AMP substrate (white shading: 0-49% inhibition at 1 μg/ml, light grey: 50-69% inhibition, grey: 70-89% inhibition, and dark grey: 90-100% inhibition). Each square, except the furthest right in each row, represents a biparatopic antibody produced by the combination of parental antibodies indicated on the horizontal and vertical axes. The furthest right square in each row represents the parental bivalent anti-CD73 antibody reconstructed using Fab-arm exchange. The bottom row (“AS30”) indicates pairings with an irrelevant antibody AS30, to produce monovalent versions of the parental antibodies.

FIG. 2 depicts the relative affinities of parental and biparatopic antibodies for CD73. Antibodies (each parental in monovalent form containing an irrelevant AS30 arm and the biparatopic antibodies) were immobilized and exposed to soluble CD73 in the flow.

FIG. 3 depicts the confirmation of biparatopic antibody formation by capillary isoelectric focusing (cIEF). Duobody products and the parental antibodies (4 μg each) were digested with IdeZ to obtain the F(ab′)2 and Fc and resolved by cIEF. For the peaks between pI 9.0 and 9.5: Right peak: E3.2(F405L) parental, Left peak: H19(K409R) parental, Middle peak: Fab-arm exchange reaction (cFAE) product E3.2/H19. The F(ab′)2 fragments have pI values above 8.5. The peaks between pI 7.5 and 8 represent the Fc's. The peak at 7.1 is IdeZ.

FIG. 4 depicts the potency dose-responses for 11 biparatopic antibodies, parental antibodies, and the mixtures of the parental antibodies. COR-L23 cells were incubated with antibodies for 3 hours followed by CD73 activity determined by an LS/MS-based assay. The activity (fraction of the no-antibody control) is plotted for the biparatopic antibodies (light grey circles), parental mixtures (black) and each of two parental antibodies (dark grey circles and squares) are shown relative to the total antibody concentration.

FIG. 5A-FIG. 5D depict epitope binning by biolayer interferometry (Octet). A mixture of CD73 with a molar excess of Fab was incubated with monovalent parental antibodies immobilized on a solid support. FIG. 5A depicts a schematic of the assay format showing the condition of non-overlapping epitopes and no blocking (top panel) or overlapping epitopes producing complete blocking of capture (bottom panel). FIG. 5B depicts the capture of CD73/Fab complexes by immobilized antibodies. The capture was normalized to the signal from CD73 alone in the absence of Fab. FIG. 5C depicts epitope binning based on the inhibition of capture. FIG. 5D depicts inhibition of CD73 activity on COR-L23 cells versus the ability of antibodies to capture a CD73/Fab complex in vitro. Grey-filled circles: capture of a CD73/Fab complex of the same antibody on the support (parental pair).

FIG. 6A-FIG. 6C depicts the structure of TB19 with CD73. FIG. 6A depicts a schematic representation of the different conformational states of CD73. The CD73 N-terminal domain of CD73 and the C-terminal domain are labeled “N” and “C” respectively. The linkers connecting the two domains are represented by grey coils. The zinc cofactors are shown as small grey spheres in the N-terminal domain. The substrate is depicted by “S”. FIG. 6B depicts two TB19 Fv domains binding one CD73 dimer in the intermediate conformation from two different angles. CD73 is colored as in FIG. 6A with the zinc and phosphate molecules shown in a black circle. TB19 Fab is shown as a light grey schematic representation. FIG. 6C depicts the mapping of the TB19 epitope residues on CD73. The same coloring scheme is utilized as in FIG. 6B with the CD73 epitope residues recognized by TB19 shown in light grey.

FIG. 7A-FIG. 7B depicts the spatial arrangement of the CD73 monomer with the TB19 Fv. FIG. 7A depicts TB19 bound to CD73 in the partially-open conformation. The zinc and inorganic phosphate at the catalytic center are labelled as “Zn” and “Pi” respectively. Interacting residues (within 4 Å) in the TB19 Fv and the N-terminal domain are shown in stick form. For illustration, the substrate analog AMPCP (in stick form) bound by the C-terminal domain of the closed conformer structure 4H21 is superimposed on the TB19:CD73 structure to show its position and the interacting CD73 residues Phe417 and Phe500. Note that AMPCP is not present in the TB19 structure. FIG. 7B depicts the modeling of TB19 onto the closed conformation structure 4H21 by superimposing the CD73 N-terminal domains of the two structures. The zinc ions and β-phosphonate of AMPCP in 4H21 occupy the same positions as the zinc and inorganic phosphate in the TB19-bound CD73 structure. The TB19 variable region clashes with the C-terminal domain in 4H21.

FIG. 8A-FIG. 8C depict the structures of TB38 with CD73. CD73 N-terminal domain is labelled “N”, CD73 C-terminal domain is labelled “C”, and the gray coil is the linker. FIG. 8A depicts a TB38 Fab::CD73 structure with CD73 in the open conformation. The TB38 Fab is shown and labelled. FIG. 8B depicts a TB38 Fv::CD73 structure with CD73 in an open/closed hybrid conformation. The TB38 Fv is shown and labelled. FIG. 8C depicts the mapping of the TB38 epitope residues on CD73 (open/closed hybrid conformation) shown and labelled.

FIG. 9A-FIG. 9B depict potential modes of co-engagement of CD73 by the TB19/TB38 biparatopic antibody. Bispecific antibodies are modeled based on TB19:CD73, TB38:CD73, and full-IgG1 (PDB 1ZHZ) structures. FIG. 9A depicts a surface representation of the TB19/TB38 biparatopic antibody. The distance between the last residues in the CH1 domains is shown as a black line. The TB19 Fab, the TB38 Fab, and the Fc are labelled. FIG. 9B depicts a model for four TB19/TB38 biparatopic antibodies bound by CD73 in the partially-open configuration. CD73 N-terminal domains and C-terminal domains are shown. The distances separating the last residues of the CH1 domains are shown as black lines.

FIG. 10 depicts the epitopes for TB19 and TB38 mapped onto one subunit of the CD73 homodimer shown in the partially open configuration as in the TB19 co-crystal structure. (dark gray/light gray).

FIG. 11A-FIG. 11C depict CD73 conformers structures associated with the concept cartoons in FIG. 6 above. FIG. 11A depicts a representation of CD73 conformations similar to that shown in FIG. 6, reflecting the key features of each conformer. FIG. 11B depicts actual structural equivalents to the diagrammatic representations in FIG. 11A. Note that the N-terminal domain on the right rotates back into the plane of the page between the open, TB19, and closed configurations. FIG. 11C depicts the structures of CD73 monomers with the C-terminal domains aligned showing the rotation of the N-terminal domain in each of the 3 conformations: open, TB-19 and closed. The TB19 Fab is not shown for clarity. The view is from below relative to those in FIG. 11B, perpendicular to the plane of rotation of the N-terminal domain. C-terminal residue of extracellular domain, zinc atoms in the closed conformer structure 4H21, and substrate analog AMPCP in 4H21 are shown.

FIG. 12 depicts Fo-Fc omit map for zincs and phosphate in the N-terminal domain structure with TB19 at 5 sigma. balls: zinc; sticks: oxygen and phosphorus of the phosphate ion.

FIG. 13A-FIG. 13B depict the raw Biacore sensorgram fits to kinetic response data to assess bivalent binding to single CD73 by biparatopic antibodies, as shown in FIG. 2 above. Fits are based on a 1:1 Langmuir binding model. Kinetic values based on these fits are shown in Table 6. Biphasic association and dissociation kinetics were observed with multiple antibodies. In those cases, (indicated by asterisks) fits were performed at two concentrations of CD73 (32 nM and 12 nM) using a reiterative process to obtain abundances and rate constants for each component during association and dissociation. FIG. 13A depicts data for monovalent parental antibodies. FIG. 13B depicts data for biparatopic antibodies.

FIG. 14 depicts epitope mapping by a premix competition approach. The capture of Fab:CD73 complexes in solution by monovalent CD73 antibodies on protein A biosensor tips was followed by Octet. Titles: name of antibody loaded on the tips. Sensorgram colors identify the Fab preincubated with CD73 (see legend). Blue trace: CD73 alone. The initial rise for 300s reflects loading of the IgGs on the tips. Following washing, CD73 premixed with excess Fab was applied. Higher responses reflect greater mass capture. Note that preincubation with a Fab with an overlapping epitope will block capture and produce no change in the sensorgram. Capture of a CD73 bound by a Fab to a non-overlapping epitope will produce a higher signal than CD73 alone on account of the larger size of the complex. The binding shift values normalized to CD73 are shown in FIG. 5B.

FIG. 15 depicts half-times for dissociation of CD73 from immobilized monovalent parent and biparatopic antibodies. Half times shown are based on the first order rate constants presented in Table 6. Where biphasic kinetics were observed the major and minor components and the fraction of each (italics) are shown. nd: not detectable.

### DETAILED DESCRIPTION

Anti-CD73 parental, monospecific antigen-binding proteins are provided. Biparatopic anti-CD73 antigen-binding proteins derived from the parental antigen-binding proteins are also provided. Methods of inhibiting CD73 activity and methods of treating CD73-mediated diseases and disorders are also provided.

Generally, nomenclature used in connection with cell and tissue culture, molecular biology, immunology, microbiology, genetics and protein and nucleic acid chemistry and hybridization described herein are those well-known and commonly used in the art. The methods and techniques provided herein are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclature used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

Unless otherwise defined herein, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

So that the invention may be more readily understood, certain terms are first defined.

### CD73

The CD73 monomer, with N- and C-terminal domains which are connected through a flexible α-helical linker, is expressed at the cell surface attached to C-terminal GPI anchor. In the physiological form, two monomers associate through extensive noncovalent contacts between the C-terminal domains forming a dimer (Heuts et al. 2012. Chembiochem: a European journal of chemical biology. 13:2384-2391; Knapp et al. 2012. Structure (London, England:1993) 20:2161-2173). The active site in each monomer of CD73 is comprised of substrate contact residues in both the N- and C-terminal domains in addition to zinc cofactors bound by the N-terminal domain (Knapp 2012, supra). Following binding of the AMP substrate to the C-terminal domain, the N-terminal domain and zinc cofactors align with the AMP in a “closed” CD73 conformation in which catalysis takes place to generate the adenosine product. A large lateral rotation of the N-terminal domain to re-expose the substrate binding site in the “open” conformer then allows product release (Knapp 2012, supra). A limited solvent access to the active site in the closed conformer indicates that cycling between the two forms is required for substrate binding and product release, i.e., efficient enzymatic activity (Knapp 2012, supra).

### Antigen-Binding Proteins

As used herein, the term “antibody” or “antigen-binding protein” refers to an immunoglobulin molecule that specifically binds to, or is immunologically reactive with an antigen or epitope, and includes both polyclonal and monoclonal antibodies, as well as functional antibody fragments, including but not limited to fragment antigen-binding (Fab) fragments, F(ab′)2 fragments, Fab′ fragments, Fv fragments, recombinant IgG (rIgG) fragments, single chain variable fragments (scFv) and single domain antibodies (e.g., sdAb, sdFv, nanobody) fragments. The term “antibody” includes genetically engineered or otherwise modified forms of immunoglobulins, such as intrabodies, peptibodies, chimeric antibodies, fully human antibodies, humanized antibodies, meditope-enabled antibodies, heteroconjugate antibodies (e.g., bispecific antibodies, diabodies, triabodies, tetrabodies, tandem di-scFv, tandem tri-scFv) and the like. Unless otherwise stated, the term “antibody” should be understood to encompass functional antibody fragments thereof.

As used herein, the term “complementarity determining region” or “CDR” refers to non-contiguous sequences of amino acids within antibody variable regions, which confer antigen specificity and binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” or “FR” are known in the art to refer to the non-CDR portions of the variable regions of the heavy and light chains. In general, there are four FRs in each heavy chain variable region (FR-H1, FR-H2, FR-H3, and FR-H4), and four FRs in each light chain variable region (FR-L1, FR-L2, FR-L3, and FR-L4).

The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme), Al-Lazikani et al., (1997) JMB 273, 927-948 (“Chothia” numbering scheme), MacCallum et al., J. Mol. Biol. 262:732-745 (1996), “Antibody-antigen interactions: Contact analysis and binding site topography,” J. Mol. Biol. 262, 732-745. (“Contact” numbering scheme), Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme), and Honegger A and Pluckthun A, “Yet another numbering scheme for immunoglobulin variable domains: an automatic modeling and analysis tool,” J Mol Biol, 2001 Jun. 8; 309(3):657-70, (AHo numbering scheme).

The boundaries of a given CDR or FR may vary depending on the scheme used for identification. For example, the Kabat scheme is based structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme.

Thus, unless otherwise specified, a “CDR” or “complementary determining region,” or individual specified CDRs (e.g., “CDR-H1”, “CDR-H2”), of a given antibody or region thereof, such as a variable region thereof, should be understood to encompass a (or the specific) complementary determining region as defined by any of the known schemes. Likewise, unless otherwise specified, an “FR” or “framework region,” or individual specified FRs (e.g., “FR-H1,” “FR-H2”) of a given antibody or region thereof, such as a variable region thereof, should be understood to encompass a (or the specific) framework region as defined by any of the known schemes. In some instances, the scheme for identification of a particular CDR or FR is specified, such as the CDR as defined by the Kabat, Chothia, or Contact method. In other cases, the particular amino acid sequence of a CDR or FR is given.

### Anti-CD73 Antigen-Binding Proteins

In one aspect, the disclosure provides antigen binding proteins with binding specificity to CD73. As used herein, “CD73” may refer to both a CD73 monomer protein or the CD73 homodimer complex formed by two non-covalently associated CD73 monomer proteins.

Exemplary anti-CD73 antigen binding protein CDRs are recited below in Table 1. Exemplary anti-CD73 antigen binding protein variable heavy (VH) and variable light (VL) domains are recited below in Table 2. Exemplary anti-CD73 antigen binding protein full length heavy and light chains are recited below in Table 3.

TABLE 1
Anti-CD73 antigen binding protein CDR sequences.

| SEQ ID NO: | Sequence | Note |
|---|---|---|
| 1 | GGSIRNNY | TB19.3 CDR-H1 |
| 2 | IYISGTT | TB19.3 CDR-H2 |
| 3 | AREHYVSGTSLDN | TB19.3_CDR-H3 |
| 4 | QSVNTNY | TB19.3_CDR-L1 |
| 5 | GTS | TB19.3_CDR-L2 |
| 6 | QQDYNLPYT | TB19.3_CDR-L3 |
| 7 | GFTFSSYG | TB38.8 CDR-H1 |
| 8 | FWYDGSNK | TB38.8 CDR-H2 |
| 9 | ARAPNWDDAFDI | TB38.8_CDR-H3 |
| 10 | SGSVSTSYY | TB38.8_CDR-L1 |
| 11 | STN | TB38.8_CDR-L2 |
| 12 | VLFMGSGIWV | TB38.8_CDR-L3 |

TABLE 2
Anti-CD73 antigen binding protein VH/VL
sequences.

| SEQ | | |
|---|---|---|
| ID NO: | Sequence | Note |
| 13 | QEQLQESGPGLVKPSETLSLTCTVSGGSIRN | TB19.3 VH |
| | NYYNWIRQPAGKGLEWIGRIYISGTTNSNP | |
| | SLKSRVTMSIDTSKNQFSLKLSSVTAADTAI | |
| | YYCAREHYVSGTSLDNWGQGTLVTVSS | |
| 14 | EIVMTQSPTTLSLSPGERATLSCRASQSVNT | TB19.3 VL |
| | NYFSWYQQKPGLTPRLLIYGTSTRATGIPA | |
| | RFSGSGSGTDFTLTISSLQPEDFGIYYCQQD | |
| | YNLPYTFGQGTYLEIK | |
| 15 | QVQLVESGGGVVQPGRSLRLSCAASGFTFS | TB38.8 VH |
| | SYGMHWVRQAPGKGLEWVAVFWYDGSN | |
| | KYYADSVKGRFTISRDNSKNTLYLQMNSL | |
| | SAEDTAVYYCARAPNWDDAFDIWGQGTM | |
| | VTVSS | |
| 16 | QTVVTQEPSFSVSPGGTVTLTCGLSSGSVST | TB38.8 VL |
| | SYYPNWYQQTPGQAPRTLIYSTNTRSSGVP | |
| | DRFSGSILGNKAALTITGAQADDESDYYCV | |
| | LFMGS GIWVFGGGTKLTVL | |

TABLE 3
Anti-CD73 antigen binding protein sequences.

| SEQ ID NO: | Sequence | Note |
|---|---|---|
| 17 | QEQLQESGPGLVKPSETLSLTCTVSGGSI | TB19.3_huIgG1_ |
| | RNNYYNWIRQPAGKGLEWIGRIYISGTT | K409R-Heavy |
| | NSNPSLKSRVTMSIDTSKNQFSLKLSSV | Chain |
| | TAADTAIYYCAREHYVSGTSLDNWGQ | |
| | GTLVTVSSASTKGPSVFPLAPSSKSTSG | |
| | GTAALGCLVKDYFPEPVTVSWNSGALT | |
| | SGVHTFPAVLQSSGLYSLSSVVTVPSSS | |
| | LGTQTYICNVNHKPSNTKVDKKVEPKS | |
| | CDKTHTCPPCPAPELLGGPSVFLFPPKP | |
| | KDTLMISRTPEVTCVVDVSHEDPEVK | |
| | FNWYVDGVEVHNAKTKPREEQYNSTY | |
| | RVVSVLTVLHQDWLNGKEYKCKVSNK | |
| | ALPAPIEKTISKAKGQPREPQVYTLPPSR | |
| | DELTKNQVSLTCLVKGFYPSDIAVEWE | |
| | SNGQPENNYKTTPPVLDSDGSFFLYSRL | |
| | TVDKSRWQQGNVFSCSVMHEALHNHY | |
| | TQKSLSLSPG | |
| 18 | QEQLQESGPGLVKPSETLSLTCTVSGGSI | TB19.3_huIgG1_ |
| | RNNYYNWIRQPAGKGLEWIGRIYISGTT | F405L-Heavy |
| | NSNPSLKSRVTMSIDTSKNQFSLKLSSV | Chain |
| | TAADTAIYYCAREHYVSGTSLDNWGQ | |
| | GTLVTVSSASTKGPSVFPLAPSSKSTSG | |
| | GTAALGCLVKDYFPEPVTVSWNSGALT | |
| | SGVHTFPAVLQSSGLYSLSSVVTVPSSS | |
| | LGTQTYICNVNHKPSNTKVDKKVEPKS | |
| | CDKTHTCPPCPAPELLGGPSVFLFPPKP | |
| | KDTLMISRTPEVTCVVDVSHEDPEVK | |
| | FNWYVDGVEVHNAKTKPREEQYNSTY | |
| | RVVSVLTVLHQDWLNGKEYKCKVSNK | |
| | ALPAPIEKTISKAKGQPREPQVYTLPPSR | |
| | DELTKNQVSLTCLVKGFYPSDIAVEWE | |
| | SNGQPENNYKTTPPVLDSDGSFLLYSKL | |
| | TVDKSRWQQGNVFSCSVMHEALHNHY | |
| | TQKSLSLSPG | |
| 19 | EIVMTQSPTTLSLSPGERATLSCRASQS | TB19.3_huIgG1_ |
| | VNTNYFSWYQQKPGLTPRLLIYGTSTR | -Light Chain |
| | ATGIPARFSGSGSGTDFTLTISSLQPEDF | |
| | GIYYCQQDYNLPYTFGQGTYLEIKRTV | |
| | AAPSVFIFPPSDEQLKSGTASVVCLLNN | |
| | FYPREAKVQWKVDNALQSGNSQESVT | |
| | EQDSKDSTYSLSSTLTLSKADYEKHKV | |
| | YACEVTHQGLSSPVTKSFNRGEC | |
| 20 | QVQLVESGGGVVQPGRSLRLSCAASGF | TB38.8_huIgG1_ |
| | TFSSYGMHWVRQAPGKGLEWVAVFW | K409R-Heavy |
| | YDGSNKYYADSVKGRFTISRDNSKNTL | Chain |
| | YLQMNSLSAEDTAVYYCARAPNWDDA | |
| | FDIWGQGTMVTVSSASTKGPSVFPLAPS | |
| | SKSTSGGTAALGCLVKDYFPEPVTVSW | |
| | NSGALTSGVHTFPAVLQSSGLYSLSSVV | |
| | TVPSSSLGTQTYICNVNHKPSNTKVDKK | |
| | VEPKSCDKTHTCPPCPAPELLGGPSVFL | |
| | FPPKPKDTLMISRTPEVTCVVVDVSHED | |
| | PEVKFNWYVDGVEVHNAKTKPREEQY | |
| | NSTYRVVSVLTVLHQDWLNGKEYKCK | |
| | VSNKALPAPIEKTISKAKGQPREPQVYT | |
| | LPPSRDELTKNQVSLTCLVKGFYPSDIA | |
| | VEWESNGQPENNYKTTPPVLDSDGSFF | |
| | LYSRLTVDKSRWQQGNVFSCSVMHEA | |
| | LHNHYTQKSLSLSPG | |
| 21 | QVQLVESGGGVVQPGRSLRLSCAASGF | TB38.8_huIgG1_ |
| | TFSSYGMHWVRQAPGKGLEWVAVFW | F405L-Heavy |
| | YDGSNKYYADSVKGRFTISRDNSKNTL | Chain |
| | YLQMNSLSAEDTAVYYCARAPNWDDA | |
| | FDIWGQGTMVTVSSASTKGPSVFPLAPS | |
| | SKSTSGGTAALGCLVKDYFPEPVTVSW | |
| | NSGALTSGVHTFPAVLQSSGLYSLSSVV | |
| | TVPSSSLGTQTYICNVNHKPSNTKVDKK | |
| | VEPKSCDKTHTCPPCPAPELLGGPSVFL | |
| | FPPKPKDTLMISRTPEVTCVVVDVSHED | |
| | PEVKFNWYVDGVEVHNAKTKPREEQY | |
| | NSTYRVVSVLTVLHQDWLNGKEYKCK | |
| | VSNKALPAPIEKTISKAKGQPREPQVYT | |
| | LPPSRDELTKNQVSLTCLVKGFYPSDIA | |
| | VEWESNGQPENNYKTTPPVLDSDGSFL | |
| | LYSKLTVDKSRWQQGNVFSCSVMHEA | |
| | LHNHYTQKSLSLSPG | |
| 22 | QTVVTQEPSFSVSPGGTVTLTCGLSSGS | TB38.8_huIgG1_ |
| | VSTSYYPNWYQQTPGQAPRTLIYSTNT | -Light Chain |
| | RSSGVPDRFSGSILGNKAALTITGAQAD | |
| | DESDYYCVLFMGSGIWVFGGGTKLTVL | |
| | GQPKAAPSVTLFPPSSEELQANKATLVC | |
| | LISDFYPGAVTVAWKADSSPVKAGVET | |
| | TTPSKQSNNKYAASSYLSLTPEQWKSH | |
| | RSYSCQVTHEGSTVEKTVAPTECS | |

In certain embodiments, the anti-CD73 antigen binding proteins of the disclosure comprise at least about 80%, at least about 80%, at least about 85%, at least about 90%, at least about 95%, at least about 96%, at least about 97%, at least about 98%, at least about 99%, or 100% sequence similarity or identity to any of the sequences of Table 1, Table 2, or Table 3.

In certain embodiments, the anti-CD73 antigen binding proteins of the disclosure bind a human CD73 polypeptide comprising the amino acid sequence of SEQ ID NO: 23, shown in Table 4 below.

In certain embodiments, the anti-CD73 antigen binding proteins of the disclosure bind an epitope of human CD73 polypeptide comprising the amino acids N96, G97, V98, E99, K121, P123, P156, F157, S159, N160, G162, T163, N164, L165, V166, F167, E168, R491, and D496 of SEQ ID NO: 23, shown in Table 4 below.

In certain embodiments, the anti-CD73 antigen binding proteins of the disclosure bind an epitope of human CD73 polypeptide comprising the amino acids P112, K119, A125, S126, S129, G130, L133, P134, Y135, K136, K180, L184, and N185 of SEQ ID NO: 24, shown in Table 4 below.

TABLE 4
Human CD73 and epitopes.

| SEQ ID NO: | Sequence | Note |
|---|---|---|
| 23 | WELTILHTNDVHSRLEQTSEDSSKCV | Human CD73 |
| | NASRCMGGVARLFTKVQQIRRAEPN | TB19.3 Epitope |
| | VLLLDAGDQYQGTIWFTVYKGAEVA | (Bold & Underlined) |
| | HFMNALRYDAMALGNHEFDNGVEG | Residues N96, G97, |
| | LIEPLLKEAKFPILSANIKAKGPLASQI | V98, E99, K121, |
| | SGLYLPYKVLPVGDEVVGIVGYTSKE | P123, P156, F157, |
| | TPFLSNPGTNLVFEDEITALOPEVDK | S159, N160, G162, |
| | LKTLNVNKIIALGHSGFEMDKLIAQK | T163, N164, L165, |
| | VRGVDVVVGGHSNTFLYTGNPPSKE | V166, F167, E168, |
| | VPAGKYPFIVTSDDGRKVPVVQAYA | R491, D496 |
| | FGKYLGYLKIEFDERGNVISSHGNPIL | |
| | LNSSIPEDPSIKADINKWRIKLDNYST | |
| | QELGKTIVYLDGSSQSCRFRECNMG | |
| | NLICDAMINNNLRHTDEMFWNHVS | |
| | MCILNGGGIRSPIDERNNGTITWENL | |
| | AAVLPFGGTFDLVQLKGSTLKKAFE | |
| | HSVHRYGQSTGEFLQVGGIHVVYDL | |
| | SRKPGDRVVKLDVLCTKCRVPSYDP | |
| | LKMDEVYKVILPNFLANGGDGFQMI | |
| | KDELLRHDSGDQDINVVSTYISKMK | |
| | VIYPAVEGRIKFS | |
| 24 | WELTILHTNDVHSRLEQTSEDSSKCV | Human CD73 |
| | NASRCMGGVARLFTKVQQIRRAEPN | TB38.8 Epitope |
| | VLLLDAGDQYQGTIWFTVYKGAEVA | (Bold & Underlined) |
| | HFMNALRYDAMALGNHEFDNGVEG | Residues P112, |
| | LIEPLLKEAKFPILSANIKAKGPLASQI | K119, A125, S126, |
| | SGLYLPYKVLPVGDEVVGIVGYTSK | S129, G130, L133, |
| | ETPFLSNPGTNLVFEDEITALQPEVDK | P134, Y135, K136, |
| | LKTLNVNKIIALGHSGFEMDKLIAQK | K180, L184, N185 |
| | VRGVDVVVGGHSNTFLYTGNPPSKE | |
| | VPAGKYPFIVTSDDGRKVPVVQAYA | |
| | FGKYLGYLKIEFDERGNVISSHGNPIL | |
| | LNSSIPEDPSIKADINKWRIKLDNYST | |
| | QELGKTIVYLDGSSQSCRFRECNMG | |
| | NLICDAMINNNLRHTDEMFWNHVS | |
| | MCILNGGGIRSPIDERNNGTITWENL | |
| | AAVLPFGGTFDLVQLKGSTLKKAFE | |
| | HSVHRYGQSTGEFLQVGGIHVVYDL | |
| | SRKPGDRVVKLDVLCTKCRVPSYDP | |
| | LKMDEVYKVILPNFLANGGDGFQMI | |
| | KDELLRHDSGDQDINVVSTYISKMK | |
| | VIYPAVEGRIKFS | |

### Biparatopic Anti-CD73 Antigen Binding Proteins

In one aspect, the disclosure provides biparatopic antigen binding proteins with binding specificity to a first CD73 epitope and a second CD73 epitope. As used herein, a “biparatopic” antigen binding protein binds two different epitopes on the same molecular target (i.e., biparatopic). In the instant disclosure, the biparatopic anti-CD73 antigen binding protein are derived from two parental, monospecific CD73 antigen binding proteins. The two parental antigen binding proteins each bind a different epitope on a CD73 molecule.

The biparatopic antigen binding proteins of the disclosure may have advantages over monospecific antigen binding proteins due to the potentially additive or synergistic effect of combining antibody specificities. The biparatopic antigen binding proteins of the disclosure may demonstrate potent CD73 inhibition when combined in biparatopic variants provided they bind non-overlapping epitopes on CD73. The biparatopic antigen binding proteins may further provide multiple mechanisms of inhibiting CD73 activity. CD73 inhibitory mechanisms may include, but are not limited to, blocking the formation of the catalytically-active CD73 conformer, binding of the intermediate partly-open inactive CD73 conformer, binding an open, closed and hybrid conformation, and crosslinking two or more CD73 dimers. A CD73 hybrid conformer is one in which one CD73 monomer is in the open conformation and the other CD73 monomer is in the closed conformation.

In certain embodiments, the biparatopic antigen-binding proteins of the disclosure comprise higher inhibitory activity of CD73 compared to one or both of the monospecific parental antibodies used to generate each biparatopic antigen-binding protein. In certain embodiments, the biparatopic antigen-binding proteins of the disclosure comprise higher inhibitory activity of CD73 compared to the combination of monospecific parental antibodies used to generate each biparatopic antigen-binding protein. Inhibition of CD73 activity may be determined by any method known in the art. In certain embodiments, CD73 activity is determined using COR-L23 cells expressing CD73, as described below in Example 1 and McManus et al. 2018. SLAS discovery: advancing life sciences R & D 23, 264-273.

In certain embodiments, the biparatopic anti-CD73 antigen-binding proteins of the disclosure bind to two different CD73 epitopes on the same CD73 molecule. The biparatopic anti-CD73 antigen-binding proteins may bind to two different CD73 epitopes on the same CD73 monomer protein. The biparatopic anti-CD73 antigen-binding proteins may bind to two different CD73 epitopes on the same CD73 homodimer protein.

In certain embodiments, the biparatopic anti-CD73 antigen-binding proteins of the disclosure bind to two different CD73 epitopes on two separate CD73 molecules. In certain embodiments, the first VH and VL domains of a biparatopic anti-CD73 antigen-binding protein bind a first CD73 epitope on a first CD73 dimer or homodimer molecule, and the second VH and VL domains bind a second CD73 epitope on a second CD73 dimer or homodimer molecule.

In certain embodiments, the biparatopic anti-CD73 antigen-binding proteins of the disclosure may be capable of crosslinking two or more CD73 dimer molecules. As used herein, “crosslinking” with antibodies may occur when a first binding site on a multivalent antibody binds a first epitope on a first target molecule while a second binding site on a multivalent antibody binds a second epitope on a second target molecule. The crosslinking of multiple target molecules through binding multiple bivalent antibodies may form higher order structures with enhanced stability. This may lead to reducing the koff rate of the crosslinked antigen-binding proteins relative to non-crosslinked antigen-binding proteins. By enhancing antigen-binding protein crosslinking, antigen-binding proteins with weak antigen-binding affinity may be employed. Certain antigen-binding proteins which possess weak binding affinity to their target antigen generally have limited utility. By combining antigen-binding proteins with weak binding affinity, the crosslinking effect of the disclosure may enhance their efficacy through the reduction of the koff rate.

### Methods of Heterodimerization of Antigen-Binding Proteins

The biparatopic CD73 antigen binding proteins of the disclosure may be formed though the heterodimerization of two parental CD73 antigen binding proteins. Any heterodimerization method known in the art may be used to form the biparatopic CD73 antigen binding proteins.

In certain exemplary embodiments, two Fc domains of an antibody or antigen-binding fragment thereof are heterodimerized through Fab arm exchange (FAE). In certain exemplary embodiments, a human non-IgG4 CH3 sequence is modified such that it does not comprise any amino acid residues which participate in the formation of disulfide bonds or covalent or stable non-covalent inter-heavy chain bonds with other peptides comprising an identical amino acid sequence of the CH3 region. Such a modified CH3 sequence may be IgG4-like. In certain embodiments, the antibody is IgG1 and is modified to be IgG4-like.

An exemplary method of FAE may include the steps comprising: a) providing a first antigen-binding construct having a first binding specificity, wherein said first antigen-binding construct comprises an IgG4-like CH3 region; b) providing a second antigen-binding construct having a second binding specificity which differs from said first binding specificity, wherein said second antigen-binding construct comprises an IgG4-like CH3 region; c) incubating said first and second antigen-binding constructs together under reducing conditions which allow the cysteines in the core hinge region to undergo disulfide-bond isomerization; and d) obtaining a bispecific antigen-binding construct.

The term “IgG4-like CH3 region” refers to a CH3 region which is identical to the CH3 of IgG4, e.g. human IgG4, or a CH3 region which is functionally equivalent to a IgG4 CH3 region. Functionally equivalent, in this context, means that the CH3 region, similar to the CH3 region of IgG4, does not form stable inter-half-molecule interactions. The formation of stable inter-half-molecules by a given CH3 region can e.g. be tested by replacing the CH3 of an IgG4 with that CH3 region and test for exchange under the conditions described in U.S. Pat. No. 9,212,230, incorporated herein by reference. If exchange is observed, then no stable inter-half-molecule interactions are formed. For example, an IgG4-like CH3 region may be a CH3 region which is equally efficient in allowing half-molecule exchange as a CH3 region from IgG4. Accordingly, an IgG4-like CH3 region may be structurally similar to the CH3 region of IgG4, e.g. more than 75%, such as more than 90% identical to the sequence of the CH3 region of IgG4. However, an IgG4-like CH3 region in the present context may in addition or alternatively be a CH3 region which structurally is not close to the CH3 region of IgG4, but has similar functional characteristics in that it does not comprise any amino acid residues which participate in the formation of disulfide bonds or covalent or stable non-covalent inter-heavy chain bonds, such as salt bridges, with other peptides comprising an identical amino acid sequence of the CH3 region. For example, an IgG4-like CH3 region can be a mutated IgG1 CH3 region in which one or more amino acid residues that are involved in inter-half-molecule CH3-CH3 interactions have been changed or deleted.

Exemplary amino acid residue modifications include R238Q, D239E, K292R, K292Y, K292F, K292W, Q302E, and P328L. Additional exemplary amino acid residue modifications include a P228S hinge mutation. Further amino acid residue modifications include F405L or K409R CH3 domain mutation. Mixing of the two antibodies with a reducing agent leads to FAE. For example, but in no way limiting, a first parental, monospecific antibody comprising an F405L modification may undergo FAE with a second parental, monospecific antibody comprising an K409R modification. This technology is described in U.S. Pat. No. 9,212,230 and Labrijn A. F. PNAS (2013) 110(13):5145-5150.

In certain exemplary embodiments, the two Fc domains of an antigen-binding construct are heterodimerized through knobs-into-holes pairing. This dimerization technique utilizes “protuberances” or “knobs” with “cavities” or “holes” engineered into the interface of CH3 domains. Where a suitably positioned and dimensioned knob or hole exists at the interface of either the first or second CH3 domain, it is only necessary to engineer a corresponding hole or knob, respectively, at the adjacent interface, thus promoting and strengthening Fc domain pairing in the CH3/CH3 domain interface. The IgG Fc domain that is fused to the VHH is provided with a knob, and the IgG Fc domain of the conventional antibody is provided with a hole designed to accommodate the knob, or vice-versa. A “knob” refers to an at least one amino acid side chain, typically a larger side chain, that protrudes from the interface of the CH3 portion of a first Fc domain. The protrusion creates a “knob” which is complementary to and received by a “hole” in the CH3 portion of a second Fc domain. The “hole” is an at least one amino acid side chain, typically a smaller side chain, which recedes from the interface of the CH3 portion of the second Fc domain. This technology is described in U.S. Pat. No. 5,821,333; Ridgway et al. Protein Engineering (1996) 9:617-621); and Carter P. J. Immunol. Methods (2001) 248: 7-15.

Exemplary amino acid residues that may act as a knob include arginine (R), phenylalanine (F), tyrosine (Y) and/or tryptophan (W). An existing amino acid residue in the CH3 domain may be replaced or substituted with a knob amino acid residue. Exemplary amino acids to substitute may include any amino acids with a small side chain, such as alanine (A), asparagine (N), aspartic acid (D), glycine (G), serine (S), threonine (T), and/or valine (V).

Exemplary amino acid residues that may act as the hole include alanine (A), serine (S), threonine (T), or valine (V). An existing amino acid residue in the CH3 domain may be replaced or substituted with a hole amino acid residue. Exemplary amino acids to substitute may include any amino acids with a large side chain, such as arginine (R), phenylalanine (F), tyrosine (Y) and/or tryptophan (W).

In certain exemplary embodiments, the CH3 domain is derived from a human IgG1 antibody. Exemplary amino acid substitutions to the CH3 domain include T366Y, T366W, F405A, F405W, Y407T, Y407A, Y407V, T394S, or combinations thereof. A particularly exemplary combination is T366Y or T366W for the knob mutation on a first CH3 domain and Y407T or Y407V for the hole mutation on a second CH3 domain.

In certain exemplary embodiments, the two Fc domains of the antigen-binding construct are heterodimerized through electrostatic steering effects. This dimerization technique utilizes electrostatic steering to promote and strengthen Fc domain pairing in the CH3/CH3 domain interface. The charge complementarity between two CH3 domains is altered to favor heterodimerization (opposite charge paring) over homodimerization (same charge pairing). In this method, the electrostatic repulsive forces prevent homodimerization.

Exemplary amino acid residue substitution may include K409D, K392D, and/or K370D in a first CH3 domain and D399K, E356K, and/or E357K in a second CH3 domain. This technology is described in US Patent Publication No. 2014/0154254 A1 and Gunasekaran K. JBC (2010) 285(25):19637-19646.

In certain exemplary embodiments, the two Fc domains of the antigen-binding construct are heterodimerized through hydrophobic interaction effects. This dimerization technique utilizes hydrophobic interactions instead of electrostatic ones to promote and strengthen Fc domain pairing in the CH3/CH3 domain interface. Exemplary amino acid residue substitution may include K409W, K360E, Q347E, Y349S, and/or S354C in a first CH3 domain, and D399V, F405T, Q347R, E357W, and/or Y349C in a second CH3 domain. Exemplary pairs of amino acid residue substitutions between a first CH3 domain and a second CH3 domain include K409W:D399V, K409W:F405T, K360E:Q347R, Y349S:E357W, and S354C:Y349C. This technology is described in US Patent Publication No. 2015/0307628 A1.

### Expression of Antigen-Binding Proteins

In one aspect, polynucleotides encoding the binding proteins (e.g., antigen-binding proteins) disclosed herein are provided. Methods of making binding proteins comprising expressing these polynucleotides are also provided.

Polynucleotides encoding the binding proteins disclosed herein are typically inserted in an expression vector for introduction into host cells that may be used to produce the desired quantity of the claimed antibodies, or fragments thereof. Accordingly, in certain aspects, the disclosure provides expression vectors comprising polynucleotides disclosed herein and host cells comprising these vectors and polynucleotides.

The term “vector” or “expression vector” is used herein to mean vectors used in accordance with the present invention as a vehicle for introducing into and expressing a desired gene in a cell. As known to those skilled in the art, such vectors may readily be selected from the group consisting of plasmids, phages, viruses and retroviruses. In general, vectors compatible with the instant invention will comprise a selection marker, appropriate restriction sites to facilitate cloning of the desired gene and the ability to enter and/or replicate in eukaryotic or prokaryotic cells.

Numerous expression vector systems may be employed for the purposes of this invention. For example, one class of vector utilizes DNA elements which are derived from animal viruses such as bovine papilloma virus, polyoma virus, adenovirus, vaccinia virus, baculovirus, retroviruses (RSV, MMTV or MOMLV), or SV40 virus. Others involve the use of polycistronic systems with internal ribosome binding sites. Additionally, cells which have integrated the DNA into their chromosomes may be selected by introducing one or more markers which allow selection of transfected host cells. The marker may provide for prototrophy to an auxotrophic host, biocide resistance (e.g., antibiotics) or resistance to heavy metals such as copper. The selectable marker gene can either be directly linked to the DNA sequences to be expressed, or introduced into the same cell by co-transformation. Additional elements may also be needed for optimal synthesis of mRNA. These elements may include signal sequences, splice signals, as well as transcriptional promoters, enhancers, and termination signals. In some embodiments, the cloned variable region genes are inserted into an expression vector along with the heavy and light chain constant region genes (e.g., human constant region genes) synthesized as discussed above.

In other embodiments, the binding polypeptides may be expressed using polycistronic constructs. In such expression systems, multiple gene products of interest such as heavy and light chains of antibodies may be produced from a single polycistronic construct. These systems advantageously use an internal ribosome entry site (IRES) to provide relatively high levels of polypeptides in eukaryotic host cells. Compatible IRES sequences are disclosed in U.S. Pat. No. 6,193,980, which is incorporated by reference herein in its entirety for all purposes. Those skilled in the art will appreciate that such expression systems may be used to effectively produce the full range of polypeptides disclosed in the instant application.

More generally, once a vector or DNA sequence encoding an antibody, or fragment thereof, has been prepared, the expression vector may be introduced into an appropriate host cell. That is, the host cells may be transformed. Introduction of the plasmid into the host cell can be accomplished by various techniques well known to those of skill in the art. These include, but are not limited to, transfection (including electrophoresis and electroporation), protoplast fusion, calcium phosphate precipitation, cell fusion with enveloped DNA, microinjection, and infection with intact virus. See, Ridgway, A. A. G. “Mammalian Expression Vectors” Chapter 24.2, pp. 470-472 Vectors, Rodriguez and Denhardt, Eds. (Butterworths, Boston, Mass. 1988). Plasmid introduction into the host can be by electroporation. The transformed cells are grown under conditions appropriate to the production of the light chains and heavy chains, and assayed for heavy and/or light chain protein synthesis. Exemplary assay techniques include enzyme-linked immunosorbent assay (ELISA), radioimmunoassay (RIA), or fluorescence-activated cell sorter analysis (FACS), immunohistochemistry and the like.

As used herein, the term “transformation” shall be used in a broad sense to refer to the introduction of DNA into a recipient host cell that changes the genotype and consequently results in a change in the recipient cell.

Along those same lines, “host cells” refers to cells that have been transformed with vectors constructed using recombinant DNA techniques and encoding at least one heterologous gene. In descriptions of processes for isolation of polypeptides from recombinant hosts, the terms “cell” and “cell culture” are used interchangeably to denote the source of antibody unless it is clearly specified otherwise. In other words, recovery of polypeptide from the “cells” may mean either from spun down whole cells, or from the cell culture containing both the medium and the suspended cells.

In one embodiment, a host cell line used for antibody expression is of mammalian origin. Those skilled in the art can determine particular host cell lines which are best suited for the desired gene product to be expressed therein. Exemplary host cell lines include, but are not limited to, DG44 and DUXB11 (Chinese Hamster Ovary lines, DHFR minus), HELA (human cervical carcinoma), CV-1 (monkey kidney line), COS (a derivative of CV-1 with SV40 T antigen), R1610 (Chinese hamster fibroblast) BALBC/3T3 (mouse fibroblast), HAK (hamster kidney line), SP2/O (mouse myeloma), BFA-1c1BPT (bovine endothelial cells), RAJI (human lymphocyte), 293 (human kidney). In one embodiment, the cell line provides for altered glycosylation, e.g., afucosylation, of the antibody expressed therefrom (e.g., PER.C6® (Crucell) or FUT8-knock-out CHO cell lines (Potelligent® cells) (Biowa, Princeton, N.J.)). In one embodiment, NSO cells may be used. CHO cells are particularly useful. Host cell lines are typically available from commercial services, e.g., the American Tissue Culture Collection, or from published literature.

In vitro production allows scale-up to give large amounts of the desired polypeptides. Techniques for mammalian cell cultivation under tissue culture conditions are known in the art and include homogeneous suspension culture, e.g. in an airlift reactor or in a continuous stirrer reactor, or immobilized or entrapped cell culture, e.g. in hollow fibers, microcapsules, on agarose microbeads or ceramic cartridges. If necessary and/or desired, the solutions of polypeptides can be purified by the customary chromatography methods, for example gel filtration, ion-exchange chromatography, chromatography over DEAE-cellulose and/or (immuno-) affinity chromatography.

Genes encoding the binding polypeptides featured in the invention can also be expressed in non-mammalian cells such as bacteria or yeast or plant cells. In this regard it will be appreciated that various unicellular non-mammalian microorganisms such as bacteria can also be transformed, i.e., those capable of being grown in cultures or fermentation. Bacteria, which are susceptible to transformation, include members of the enterobacteriaceae, such as strains of Escherichia coli or Salmonella; Bacillaceae, such as Bacillus subtilis; Pneumococcus; Streptococcus, and Haemophilus influenzae. It will further be appreciated that, when expressed in bacteria, the polypeptides can become part of inclusion bodies. The polypeptides must be isolated, purified and then assembled into functional molecules.

In addition to prokaryotes, eukaryotic microbes may also be used. Saccharomyces cerevisiae, or common baker's yeast, is the most commonly used among eukaryotic microorganisms, although a number of other strains are commonly available. For expression in Saccharomyces, the plasmid YRp7, for example (Stinchcomb et al., Nature, 282:39 (1979); Kingsman et al., Gene, 7:141 (1979); Tschemper et al., Gene, 10:157 (1980)), is commonly used. This plasmid already contains the TRP1 gene which provides a selection marker for a mutant strain of yeast lacking the ability to grow in tryptophan, for example ATCC No. 44076 or PEP4-1 (Jones, Genetics, 85:12 (1977)). The presence of the trpl lesion as a characteristic of the yeast host cell genome then provides an effective environment for detecting transformation by growth in the absence of tryptophan.

### Methods of Administering Antigen-Binding Proteins

Methods of preparing and administering binding proteins (e.g., antigen-binding proteins disclosed herein) to a subject are well known to or are readily determined by those skilled in the art. The route of administration of the binding proteins of the current disclosure may be oral, parenteral, by inhalation or topical. The term parenteral as used herein includes intravenous, intraarterial, intraperitoneal, intramuscular, subcutaneous, rectal or vaginal administration. While all these forms of administration are clearly contemplated as being within the scope of the current disclosure, a form for administration would be a solution for injection, in particular for intravenous or intraarterial injection or drip. Usually, a suitable pharmaceutical composition for injection may comprise a buffer (e.g. acetate, phosphate or citrate buffer), a surfactant (e.g. polysorbate), optionally a stabilizer agent (e.g. human albumin), etc. However, in other methods compatible with the teachings herein, the modified antibodies can be delivered directly to the site of the adverse cellular population thereby increasing the exposure of the diseased tissue to the therapeutic agent.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. In the compositions and methods of the current disclosure, pharmaceutically acceptable carriers include, but are not limited to, 0.01-0.1 M or 0.05M phosphate buffer, or 0.8% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives may also be present such as for example, antimicrobials, antioxidants, chelating agents, and inert gases and the like. More particularly, pharmaceutical compositions suitable for injectable use include sterile aqueous solutions (where water soluble) or dispersions and sterile powders for the extemporaneous preparation of sterile injectable solutions or dispersions. In such cases, the composition must be sterile and should be fluid to the extent that easy syringability exists. It should be stable under the conditions of manufacture and storage, and should also be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants.

Prevention of the action of microorganisms can be achieved by various antibacterial and antifungal agents, for example, parabens, chlorobutanol, phenol, ascorbic acid, thimerosal and the like. Isotonic agents, for example, sugars, polyalcohols, such as mannitol, sorbitol, or sodium chloride may also be included in the composition. Prolonged absorption of the injectable compositions can be brought about by including in the composition an agent which delays absorption, for example, aluminum monostearate and gelatin.

In any case, sterile injectable solutions can be prepared by incorporating an active compound (e.g., a modified binding polypeptide by itself or in combination with other active agents) in the required amount in an appropriate solvent with one or a combination of ingredients enumerated herein, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, methods of preparation typically include vacuum drying and freeze-drying, which yield a powder of an active ingredient plus any additional desired ingredient from a previously sterile-filtered solution thereof. The preparations for injections are processed, filled into containers such as ampoules, bags, bottles, syringes or vials, and sealed under aseptic conditions according to methods known in the art. Further, the preparations may be packaged and sold in the form of a kit such as those described in co-pending U.S. Ser. No. 09/259,337 and U.S. Ser. No. 09/259,338 each of which is incorporated herein by reference. Such articles of manufacture can include labels or package inserts indicating that the associated compositions are useful for treating a subject suffering from, or predisposed to autoimmune or neoplastic disorders.

Effective doses of the compositions of the present disclosure, for the treatment of the above described conditions vary depending upon many different factors, including means of administration, target site, physiological state of the patient, whether the patient is human or an animal, other medications administered, and whether treatment is prophylactic or therapeutic. Usually, the patient is a human, but non-human mammals including transgenic mammals can also be treated. Treatment dosages may be titrated using routine methods known to those of skill in the art to optimize safety and efficacy.

For passive immunization with a binding polypeptide, the dosage can range, e.g., from about 0.0001 to 100 mg/kg, and more usually 0.01 to 5 mg/kg (e.g., 0.02 mg/kg, 0.25 mg/kg, 0.5 mg/kg, 0.75 mg/kg, 1 mg/kg, 2 mg/kg, etc.), of the host body weight. For example, dosages can be 1 mg/kg body weight or 10 mg/kg body weight or within the range of 1-10 mg/kg, e.g., at least 1 mg/kg. Doses intermediate in the above ranges are also intended to be within the scope of the current disclosure. Subjects can be administered such doses daily, on alternative days, weekly or according to any other schedule determined by empirical analysis. An exemplary treatment entails administration in multiple dosages over a prolonged period, for example, of at least six months. Additional exemplary treatment regimens entail administration once per every two weeks or once a month or once every 3 to 6 months. Exemplary dosage schedules include 1-10 mg/kg or 15 mg/kg on consecutive days, 30 mg/kg on alternate days or 60 mg/kg weekly. In some methods, two or more binding proteins with different binding specificities are administered simultaneously, in which case the dosage of each antibody administered falls within the ranges indicated.

Binding proteins described herein can be administered on multiple occasions. Intervals between single dosages can be weekly, monthly or yearly. Intervals can also be irregular as indicated by measuring blood levels of modified binding polypeptide or antigen in the patient. In some methods, dosage is adjusted to achieve a plasma modified binding polypeptide concentration of 1-1000 μg/ml and in some methods 25-300 μg/ml. Alternatively, binding polypeptides can be administered as a sustained release formulation, in which case less frequent administration is required. For antibodies, dosage and frequency vary depending on the half-life of the antibody in the patient. In general, humanized antibodies show the longest half-life, followed by chimeric antibodies and nonhuman antibodies.

The dosage and frequency of administration can vary depending on whether the treatment is prophylactic or therapeutic. In prophylactic applications, compositions containing the present antibodies or a cocktail thereof are administered to a patient not already in the disease state to enhance the patient's resistance. Such an amount is defined to be a “prophylactic effective dose.” In this use, the precise amounts again depend upon the patient's state of health and general immunity, but generally range from 0.1 to 25 mg per dose, especially 0.5 to 2.5 mg per dose. A relatively low dosage is administered at relatively infrequent intervals over a long period of time. Some patients continue to receive treatment for the rest of their lives. In therapeutic applications, a relatively high dosage (e.g., from about 1 to 400 mg/kg of antibody per dose, with dosages of from 5 to 25 mg being more commonly used for radioimmunoconjugates and higher doses for cytotoxin-drug modified antibodies) at relatively short intervals is sometimes required until progression of the disease is reduced or terminated, or until the patient shows partial or complete amelioration of disease symptoms. Thereafter, the patient can be administered a prophylactic regime.

Binding polypeptides described herein can optionally be administered in combination with other agents that are effective in treating the disorder or condition in need of treatment (e.g., prophylactic or therapeutic). Effective single treatment dosages (i.e., therapeutically effective amounts) of ⁹⁰Y-labeled modified antibodies of the current disclosure range from between about 5 and about 75 mCi, such as between about 10 and about 40 mCi. Effective single treatment non-marrow ablative dosages of ¹³¹I-modified antibodies range from between about 5 and about 70 mCi, such as between about 5 and about 40 mCi. Effective single treatment ablative dosages (i.e., may require autologous bone marrow transplantation) of ¹³¹I-labeled antibodies range from between about 30 and about 600 mCi, such as between about 50 and less than about 500 mCi. In conjunction with a chimeric antibody, owing to the longer circulating half-life vis-a-vis murine antibodies, an effective single treatment non-marrow ablative dosage of ¹³¹I labeled chimeric antibodies ranges from between about 5 and about 40 mCi, e.g., less than about 30 mCi. Imaging criteria for, e.g., an ¹¹¹In label, are typically less than about 5 mCi.

While the binding polypeptides may be administered as described immediately above, it must be emphasized that in other embodiments binding polypeptides may be administered to otherwise healthy patients as a first line therapy. In such embodiments the binding polypeptides may be administered to patients having normal or average red marrow reserves and/or to patients that have not, and are not, undergoing one or more other therapies. As used herein, the administration of modified antibodies or fragments thereof in conjunction or combination with an adjunct therapy means the sequential, simultaneous, coextensive, concurrent, concomitant, or contemporaneous administration or application of the therapy and the disclosed antibodies. Those skilled in the art will appreciate that the administration or application of the various components of the combined therapeutic regimen may be timed to enhance the overall effectiveness of the treatment. A skilled artisan (e.g. an experienced oncologist) would be readily be able to discern effective combined therapeutic regimens without undue experimentation based on the selected adjunct therapy and the teachings of the instant specification.

As previously discussed, the binding polypeptides of the present disclosure, immunoreactive fragments or recombinants thereof may be administered in a pharmaceutically effective amount for the in vivo treatment of mammalian disorders. In this regard, it will be appreciated that the disclosed binding polypeptides will be formulated to facilitate administration and promote stability of the active agent.

Pharmaceutical compositions in accordance with the present disclosure typically include a pharmaceutically acceptable, non-toxic, sterile carrier such as physiological saline, nontoxic buffers, preservatives and the like. For the purposes of the instant application, a pharmaceutically effective amount of the modified binding polypeptide, immunoreactive fragment or recombinant thereof, conjugated or unconjugated to a therapeutic agent, shall be held to mean an amount sufficient to achieve effective binding to an antigen and to achieve a benefit, e.g., to ameliorate symptoms of a disease or disorder or to detect a substance or a cell. In the case of tumor cells, the modified binding polypeptide will typically be capable of interacting with selected immunoreactive antigens on neoplastic or immunoreactive cells and provide for an increase in the death of those cells. Of course, the pharmaceutical compositions of the present disclosure may be administered in single or multiple doses to provide for a pharmaceutically effective amount of the modified binding proteins.

In keeping with the scope of the present disclosure, the binding proteins of the disclosure may be administered to a human or other animal in accordance with the aforementioned methods of treatment in an amount sufficient to produce a therapeutic or prophylactic effect. The binding polypeptides of the disclosure can be administered to such human or other animal in a conventional dosage form prepared by combining the antibody of the disclosure with a conventional pharmaceutically acceptable carrier or diluent according to known techniques. It will be recognized by one of skill in the art that the form and character of the pharmaceutically acceptable carrier or diluent is dictated by the amount of active ingredient with which it is to be combined, the route of administration and other well-known variables. Those skilled in the art will further appreciate that a cocktail comprising one or more species of binding polypeptides described in the current disclosure may prove to be particularly effective.

It will be readily apparent to those skilled in the art that other suitable modifications and adaptations of the methods described herein may be made using suitable equivalents without departing from the scope of the embodiments disclosed herein. Having now described certain embodiments in detail, the same will be more clearly understood by reference to the following examples, which are included for purposes of illustration only and are not intended to be limiting.

### EXAMPLES

### Example 1— Experimental Procedures

Generation of Biparatopic Antibodies

CD73-specific monoclonal antibodies were isolated using common mouse immunization and phage display approaches using soluble human CD73 as antigen (data not shown). Twelve sequence-unrelated parental antibodies with IC50 in the range of 1-25 nM and with at least 50% inhibition of CD73 in cell based assays at saturating concentrations of antibody were selected for the study.

Bispecific variants were produced using a modification of a published Duobody procedure (Gramer et al. 2013. mAbs 5, 962-973) except using microdialysis for product purification. Equimolar amounts of F405L and K409R Fc variants of each parental huIgG1 (25-50 μg each) were combined in a total volume of 90 μL PBS to which 10 μL 7.5M mercaptoethylamine (MEA) pH7.4 was added. The mixture was incubated 4 h at 30° C. in a forced-air incubator, transferred to individual cassettes taken from 96-well dialysis plate strips (Pierce) and subjected to three rounds of dialysis (1 h, 1.5 h, and overnight) at room temperature. For more than 6 samples, the reactions were transferred to dialysis cassette strips mounted on a carrier plate. The plate was suspended over a reservoir and transferred between reservoirs containing fresh PBS after each round of dialysis. After the second dialysis, total free thiol in the retentate was below the limit of detection using DTNB. The final products were stored at 4° C. Product formation was determined by cIEF. Parental antibodies for analysis were reconstructed by crossing the F405L and K409R parents in the same fashion as the test duobodies.

Characterization of Biparatopic Antibodies

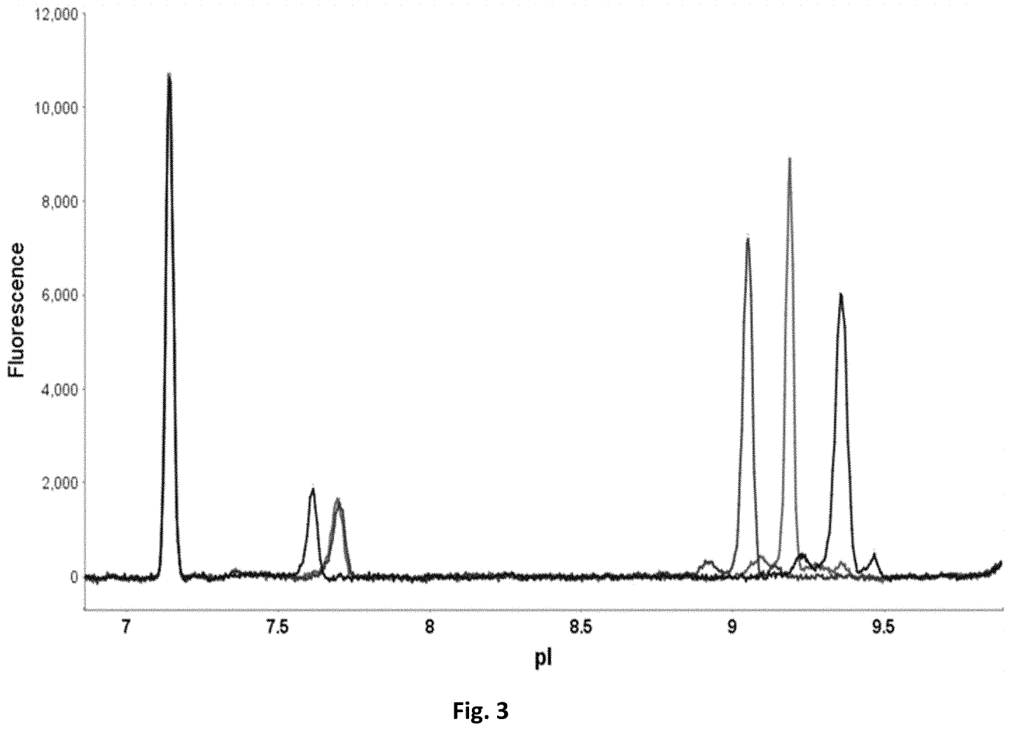

Formation of the duobody products of the Fab-arm exchange reaction (cFAE) was determined using a capillary isoelectric focusing (cIEF) (Maurice, Protein Simple, San Jose Calif.). This approach was chosen since the pI of the bispecific daughter molecules would be expected to fall between that of each of the two parents. To increase the relative contribution of charge differences in the CDRs and frameworks, cIEF was performed on F(ab′)2 fragments obtained by IdeZ digestion of the cFAE products. The cFAE product (4 μL, 1 mg/mL) was mixed with 4 μL 1 U/μL IdeZ (Fabricator Z, Genovis) in water and mixed by trituration. The tubes were incubated for 4 hours at 37° C. in an air incubator followed by addition of 36 μL 1.1× Pharmalyte methylcellulose/ampholine mixture, mixed and centrifuged 4 minutes at 13kG. The supernatant (30 μL) was transferred to a 96-well plate for analysis. Samples were loaded on a cIEF cassette for 55 seconds and focused for 1.5 minutes at 1.5 kV then 6 minutes at 3 kV. Resolved products were detected by fluorescence. Formation of the desired duobody product was assessed by the disappearance of the parental antibody F(ab′)2 peaks and formation of a F(ab′)2 peak with a pI near the average of the two parental F(ab′)2 along with the absence of a F405L parental Fc peak at ˜pI 7.6. The duobody Fc fragment with both mutations (F405L:K409R) was not resolved from the K409R parent, likely due to a limited change in the pKa of the arginine in the environment surrounding this residue. The IdeZ focused at pI 7.14 and below. An example result is shown in FIG. 3.

Analysis of Biparatopic Binding

The ability of the biparatopic antibodies to engage CD73 bivalently (e.g., at two epitopes) was determined by comparing the affinity of the biparatopic antibodies with monovalent antibodies using surface plasmon resonance (SPR). Monovalent antibodies were used to prevent bivalent interactions with CD73. SPR was performed on a Biacore T200 instrument (GE Healthcare) at 25° C. using HBS-EP+ (10 mM HEPES, 150 mM NaCl, 3 mM EDTA, 0.05% (v/v) surfactant P20, pH 7.4) as running buffer and Protein A series S sensor chips (GE Healthcare). To minimize avidity effects from the binding of CD73 by separate monovalent antibodies on the chip, binding was measured at very low response (less than 10 RU). Antibodies were diluted to limit capture to between 5 and 30 RU during a 30 second injection at 10 μL/min. Multiple concentrations of CD73 (32, 12, and 3 nM) were then passed over the captured antibodies for 5 minutes at 30 μL/min. Dissociation was measured for 30 minutes. The sensor surface was then regenerated with 10 mM glycine-HCl pH 1.5 for 30 sec at 20 μL/min. Kinetic constants were calculated using a 1:1 Langmuir binding model using the Biacore T200 Evaluation software (GE Healthcare). The 1:1 Langmuir binding model was not used for cases in which bivalent fits using BiaEvaluation software showed lower apparent residuals, raising the possibility of biphasic binding. In those cases, kd values of each component during dissociation were determined beginning by fitting the longer half-life component to a first-order decay defined by the kinetics after 1000 seconds. An exponential fit to the residual for that component between 100-200 seconds was used to calculate the abundance and kd for rapidly dissociating component(s). The criterion that the interval used for fitting the slower component began after a minimum of four times the t1/2 of the rapidly dissociating component was applied. The components of association were separately derived by initially fitting the approach to saturation (RUmax) within a window from 100 seconds out to a point a minimum of 0.2 RU from the RUmax as a first-order reaction for a range of assumed RUmax values. The best fit parameters and RUmax were then used as a starting point for further refinement. The positive residual between observed RU and this fit extended to earlier times was treated as an independent pseudo first order reaction reflecting a rapid-binding component. A reiterative process varying the rate constants and fraction of each component with the level of binding (RU) after 300 seconds was used to obtain fits within 0.2 of the observed RU and a near-zero slope for the net residual over the 300 seconds measurement. Variation testing showed the values were true R² minima for the overall fit. The RUmax had a negligible effect on the rate constants or fraction of each component. Fits were performed in Excel.

CD73 inhibition (potency) cell-based assay

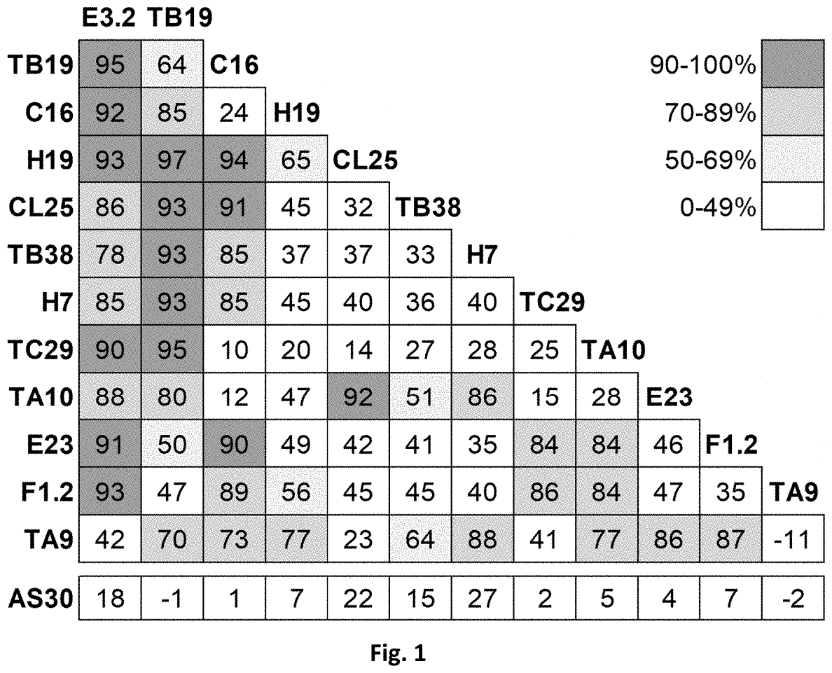

Potency of the biparatopic antibodies were determined using a modification of a previously disclosed method (McManus et al. 2018. SLAS discovery: advancing life sciences R & D 23, 264-273). COR-L23 cells expressing CD73 (4×103/well) were grown overnight to ˜50% confluence in 40 μL 1640 medium with L-glutamine and 10% heat-inactivated FBS in a 384-well transparent-bottom plate (Greiner Bio One). Antibodies diluted in 1640 medium (10 μL) were added and the plates incubated for 3 h at 37° C. Antibody dilutions and additions were performed on an Agilent Bravo liquid handler. AMPCP (100 μM) was substituted for antibodies as a zero-activity control (23,25). Substrate (5 μL 200 μM ¹⁵N₅-AMP, Silantes GmbH Munich Germany) was added using a GNF dispenser II (GNF Systems, San Diego Calif.) and the plates incubated at 37° C. for 1 h. The reactions were then quenched with 5 μL 12% formic acid in 1640 medium and a portion of the quenched reactions (40 μL) was filtered by centrifugation for 30 min at 3.5kG through a 10 kDa MWCO ultrafiltration plate (Pall). The filtrates were stored at −80° C. The adenosine product was determined by LC/MS/MS analysis as described previously (23). Data were analyzed by nonlinear least squares fits (GraphPad Prism). Activity relative to no-antibody controls in the same plate sector and normalized to the least-squares fit maximum activity (% CNTL) is shown. The results of potency determinations (Table 5) are expressed as the projected maximal % inhibition at saturating antibody concentrations. In initial screening, three concentrations (0.25, 0.5 and 1 μg/mL) were tested in quadruplicate dilution series and the average % inhibition shown (FIG. 1) is based on residual activity at a single concentration (1 μg/ml).

Epitope Binning

Epitope binning of a subset of antibodies was performed using a pre-mix format and biolayer interferometry (BLI) using a modification of a previously-described method (Abdiche et al. 2009. Analytical biochemistry 386, 172-180). In this format, binding of antigen pre-mixed with a molar excess of Fab is compared to the binding of antigen alone. Analysis was performed in 16-channel mode on an Octet QK384 (Pall Life Sciences). Antibodies were bound by protein A biosensors for 5 min, a baseline established for 1 min, then transferred to 100 nM CD73 or 100 nM CD73 with a 4-fold molar excess Fab for 3 min followed by transfer to buffer to follow dissociation for 3 min. All samples were diluted in PBS pH 7.4 containing 0.1% (w/v) bovine serum albumin and 0.01% (v/v) Tween 20 and the assays were carried out at 30° C. Data was analyzed using the ForteBio Data Analysis 7.1 software (Pall Life Sciences) by taking report points at the end of the association phase. Normalized capture values were calculated by the signal (nm) divided by the signal from CD73 alone times the relative mass of CD73 compared to the CD73::(Fab)2 complex (0.56).

Structure Determinations

Recombinant TB19 and TB38 Fab were expressed in Expi293F cells, purified by a CaptureSelect CH1-XL Affinity Matrix (ThermoFisher), and buffer exchanged into PBS. Human CD73 27-549 was cloned with a C-terminal His6-tag and expressed in ExpiHEK293 cells. CD73 was purified using a nickel column, buffer exchanged into PBS, deglycosylated overnight with PNGaseF, and further purified using size-exclusion chromatography. The molar mass of the product was determined by SEC on a Superdex 200 column in 150 mM NaCl, 20 mM HEPES pH 7.0, using multi-angle light scattering (WYATT miniDAWN® Treos and a Wyatt Optilab® T-rEX online refractometer). Data were evaluated using Wyatt ASTRA 6.1 software. Each respective Fab was then incubated with CD73 on ice for 1 hour and loaded on to a Superdex 200 10/300 GL column (GE Healthcare) pre-equilibrated with 20 mM HEPES pH 7.0, 150 mM NaCl. Fractions corresponding to the eluted complex peak were pooled and concentrated to 9 mg/ml for crystallization trials. TB19 Fab::CD73 crystallized in 0.1M sodium potassium phosphate pH 6.2, 35% 5-methyl-2,4-pentanediol, and 2.5% pentaerythritol ethoxylate at 4° C. These crystals were cryoprotected in 20% ethylene glycol and mother liquor. X-ray diffraction data was collected at EMBL Hamburg P14 using an Eiger 16M detector. Data were indexed/integrated using XDS and scaled using Aimless (Evans et al. 2013. Acta crystallographica. Section D, Biological crystallography 69, 1204-1214; Kabsch et al. 2010. Acta crystallographica. Section D, Biological crystallography 66, 125-132). Molecular replacement was performed using Phaser (McCoy et al. 2007. Journal of applied crystallography 40, 658-674) and three search ensembles: separated CD73 N- and C-terminal domains (PDB: 4H2I) and a TB19.3 Fv model generated by MOE (Molecular Operating Environment (MOE) 2013.8 Ed., Chemical Computing Group). TB38 Fab::CD73 produced crystals at 4° C. in 1.6M sodium phosphate monobasic monohydrate, 0.4M potassium phosphate dibasic, and 0.1M sodium phosphate citrate pH 5.3. Crystals were flash frozen in liquid nitrogen using 20% glycerol in mother liquor as cryoprotectant. X-ray diffraction data was collected at the European Synchrotron Radiation Facility Beamline ID-30b with a Pilatus 3 6M detector. Data were indexed/integrated using XDS and scaled using Aimless (Evans supra; Kabsch supra). Molecular replacement was performed iteratively using Phaser (McCoy supra). For the first round of molecular replacement, CD73 monomer (PDB: 4H2F) and a TB38 Fab MOE-generated model was used as search models for MOE. For the second round, the previously found CD73 monomer was separated into its N and C-terminal domains and searched along with the Fv domain alone of TB38. For both structures, model rebuilding was performed in Coot (Emsley et al. 2010. Acta crystallographica. Section D, Biological crystallography 66, 486-501) and refinement was completed using Phenix (Adams et al. 2010. Acta crystallographica. Section D, Biological crystallography 66, 213-221). Data collection and refinement statistics are listed (Table 7). Software used in this project was accessed through the SBGrid consortium (Morin et al. 2013. eLife 2, e01456).

### Example 2— Generation of Biparatopic Antibodies

A panel of biparatopic antibodies against CD73 were generated using Fab-arm exchange (cFAE) representing the pairwise combinations of 11 parental antibodies unrelated by sequence and previously showing >50% inhibition of CD73 activity in cell-based assays. Each Fab was expressed as a fusion with human IgG1 Fc containing either the F405L or K409R mutation which destabilize the parental Fc and stabilize the Fc of the biparatopic duobody product (Gramer et al. 2013. mAbs 5, 962-973; Labrijn et al. 2013. PNAS 110, 5145-5150; Labrijn et al. 2014. Nat. Protoc. 9, 2450-2463). Parental antibodies were expressed in small scale cultures, purified using protein A and recombined by cFAE. Production of the desired products was verified by cIEF (FIG. 3). Out of 121 (11×11) possible combinations, 88 biparatopic variants were generated which covered all possible combinations in at least one orientation. Eleven monospecific parental antibodies were also reconstructed as comparators by combining the parental F405L and K409R Fc variants to control for possible effect of the Fc mutations on antibody structure and function. In addition, 21 pairings were generated in both Fc orientations to control for possible positional effects of the mutations

### Example 3— Potency of Parental and Biparatopic Antibodies for Inhibiting Cellular CD73 Enzymatic Activity

Purified parental and biparatopic antibodies were tested for potency at 1 μg/mL on COR-L23 lung carcinoma cells expressing human CD73, and the product adenosine quantitated by a LC/MS based assay (McManus et al. 2018. SLAS discovery: advancing life sciences R & D 23, 264-273). The percentage of inhibition of CD73 enzymatic activity by the biparatopic antibodies at 1 nM is shown in FIG. 1. Although the extent of inhibition varied widely, most of the biparatopic combinations exhibited higher potency than either parental antibody in the form of a duobody. A number of the parental antibodies yielded highly potent daughter biparatopic variants showing ≥90% inhibition when combined with more than one other antibody. Of these, TB19 and E3.2 formed the highest number of variants with ≥90% inhibition and several of the TB19 pairs, including those with E3.2, H19, TB38 or TC29, achieved ≥95% inhibition. The TB19 and E3.2 antibodies also combined with several other antibodies to achieve ≥80% inhibition. Although both these antibodies showed this promiscuous pairing capability, they were distinguished from each other by complementarity in their pairing patterns. No major differences in the extent of inhibition were observed between biparatopic variants tested in both Fc orientations (in total 16), indicating that the position of the duobody mutations in the Fc did not significantly influence the outcome (data not shown).

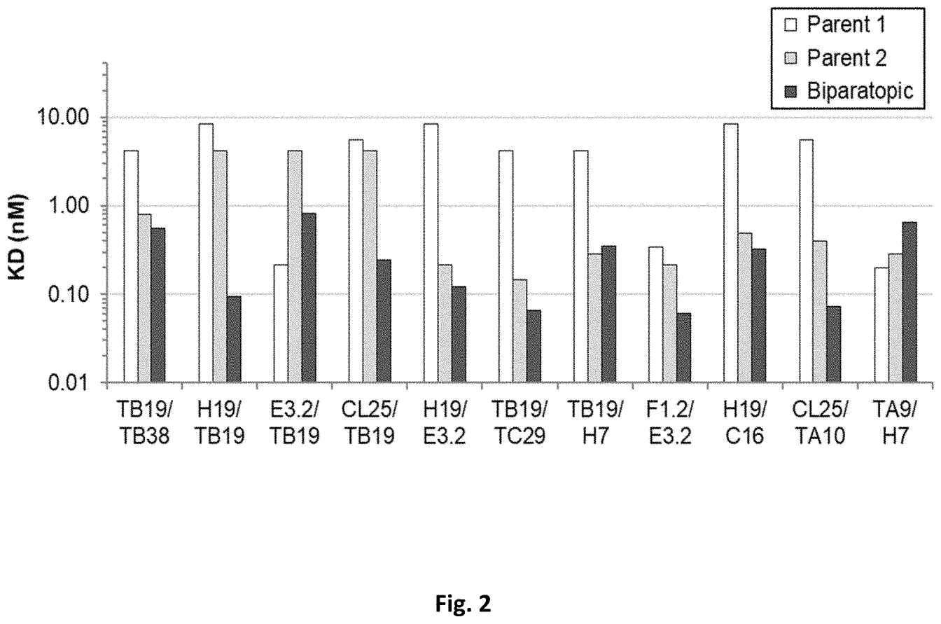

To assess whether both parental Fabs were necessary for potency, the parental antibodies were also crossed with an irrelevant antibody (AS30) to create monovalent variant IgGs with only a single Fab capable of interacting with CD73. All of these antibodies showed negligible potency, demonstrating that the Fabs from two cognate parental antibodies must participate (FIG. 1). To determine whether this was due to lower affinity, the affinity of the monovalent molecules of the most potent biparatopic antibodies to that of the biparatopic variants of which they were a part was compared. Antibodies were first bound to a solid support and binding of soluble CD73 dimer in solution was followed by SPR (Experimental Procedures described above). As seen in FIG. 2, in most cases the affinity of the biparatopic variant (KD) was similar to the affinity of the more affine parental antibody, indicating its affinity was attributable to the binding of that Fab alone. Only in two cases (H19/TB19 and CL25/TB19) did the biparatopic variant show a significantly higher affinity than either parental antibody (˜15-fold lower KD in both cases) suggesting synergistic effects, potentially due to bivalent binding to the CD73 dimer or conformational effects promoting binding. However, none of the parental antibodies in these two cases produced a similar enhancement when combined with other antibodies, suggesting a conformational effect as being less likely. Since the affinity of most of the monovalent antibodies for individual CD73 dimers was not increased by the addition of the second cognate Fab in spite of it being necessary for potency suggests that interaction of the biparatopic IgG with more than a single CD73 is required for potent inhibition.

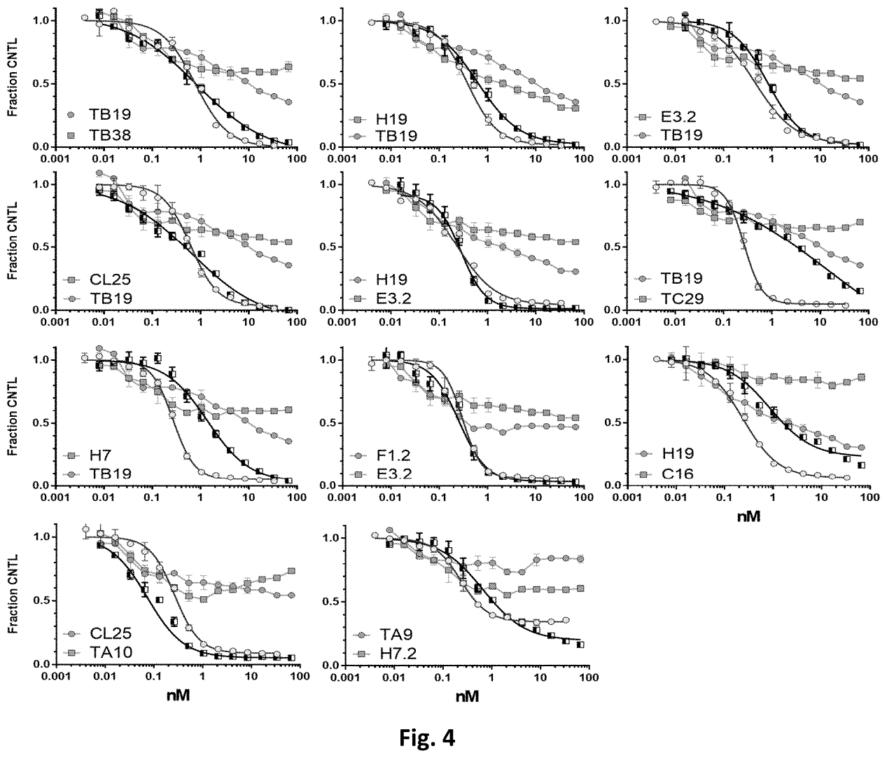

To further evaluate the benefit of combining these antibodies in biparatopic format, the EC50 and maximum inhibition at saturating antibody concentrations was determined for the most active biparatopic antibodies, along with their parental mAbs either alone or in a mixture on COR-L23 cells (Table 5, FIG. 4). In agreement with the results in FIG. 1, each biparatopic was more potent than either of their two parental antibodies that showed only partial inhibition up to 10 nM. EC50 values for all of the biparatopic antibodies were in the range of 0.2-0.8 nM. In most cases, mixtures of the parental antibodies yielded similar maximal inhibition as the biparatopic antibodies, but in half of the tested combinations, the biparatopic variant also showed a lower EC50. In the most striking case (TB19/TC29), the biparatopic showed a 50-fold lower EC50 than the antibody mixture in spite of a nearly identical affinity of the more affine TC29 monovalent parent and the biparatopic for CD73 (FIG. 2). In only a single case (CL25/TA10) was the mixture more potent (˜4-fold), indicating that the interactions with CD73 provided by that mixture could not be replicated with the biparatopic antibody.

TABLE 5
Potency of biparatopic antibodies and parental mixtures
against CD73 on COR-L23 cells. EC50 and maximum extents
of inhibition are based on nonlinear regression analysis.

| | Biparatopic | | Parental Mix | |
|---|---|---|---|---|
| | EC50 | Max | EC50 | Max |
| Parentals | (nM) | Inhib. | (nM) | Inhib. |
| TB19/TB38 | 0.777 | 100% | 0.841 | 106%† |
| H19/TB19 | 0.382 | 98% | 0.629 | 98% |
| E3.2/TB19 | 0.443 | 97% | 0.811 | 98% |
| CL25/TB19 | 0.619 | 97% | 0.636 | 109%† |
| H19/E3.2 | 0.224 | 96% | 0.283 | 99% |
| TB19/TC29 | 0.264 | 95% | 13.0 | 137%† |
| H7/TB19 | 0.270 | 95% | 0.541 | 95% |
| F1.2/E3.2 | 0.305 | 93% | 0.256 | 97% |
| H19/C16 | 0.239 | 93% | 0.863 | 77% |
| CL25/TA10 | 0.266 | 91% | 0.073 | 95% |
| TA9/H7 | 0.229 | 66% | 0.658 | 80% |

*Maximum Inhibition
†extrapolated value

The affinity of the biparatopic antibodies for CD73 was compared to that of the parental antibodies in monovalent form. To avoid potential avidity effects from binding of CD73 in solution by separate antibodies on the chip surface, parental antibodies were loaded on the chip at the lowest level sufficient to reliably assess kinetic constants. Data are grouped as shown in FIG. 2. Note that kinetic parameters for parental antibodies that are shared between multiple biparatopic antibodies are shown in each case to facilitate comparisons. Values represent fits to curves obtained with 3, 12 and 32 nM CD73 in the flow. The association rate constants in the case of biphasic kinetics are shown with their abundances following a 300 second binding phase in parentheses. Dissociation rate constant abundances are based on the To intercept of the fits to each component. Table 6 below shows the binding data that was used to generate FIG. 2.

TABLE 6
Representative SPR kinetic data for assessing bivalent engagement of single CD73.

| | | kₐ (1/Ms) | | kd (1/s) | | |
|---|---|---|---|---|---|---|
| | Name | Langmuir 1:1 | Biphasic | Langmuir 1:1 | Biphasic | K_{D} (M)* |
| Parent 1 | TB19/AS30 | 1.65E+05 | — | 6.90E−04 | | 4.18E−09 |
| Parent 2 | TB38/AS30 | 2.37E+05 | — | 1.87E−04 | | 8.14E−10 |
| Biparatopic | TB19/TB38 | 1.84E+05 | — | 1.02E−04 | | 5.55E−10 |
| Parent 1 | TB19/AS30 | 1.65E+05 | — | 6.90E−04 | | 4.18E−09 |
| Parent 2 | H19/AS30 | 6.31E+05 | — | 5.23E−03 | | 8.29E−09 |
| Biparatopic | TB19/H19 | 4.49E+05 | 2.81E+05 (65%) | 1.12E−04 | 2.70E−05 (85%) | 6.02E−11 |
| | | | 1.38E+06 (35%) | | 2.70E−03 (15%) | |
| Parent 1 | E3.2/AS30 | 5.80E+05 | 3.34E+05 (60%) | 1.72E−04 | 7.22E−05 (92%) | 1.25E−10 |
| | | | 1.22E+06 (40%) | | 1.58E−03 (8%) | |
| Parent 2 | TB19/AS30 | 1.65E+05 | — | 6.90E−04 | — | 4.18E−09 |
| Biparatopic | E3.2/TB19 | 3.64E+05 | 1.58E+05 (57%) | 1.64E−04 | 1.28E−04 (91%) | 3.52E−10 |
| | | | 8.50E+05 (43%) | | 4.06E−03 (9%) | |
| Parent 1 | CL25/AS30 | 5.54E+05 | — | 3.06E−03 | | 5.52E−09 |
| Parent 2 | TB19/AS30 | 1.65E+05 | — | 6.90E−04 | | 4.18E−09 |
| Biparatopic | CL25/TB19 | 4.93E+05 | 3.24E+05 (70%) | 1.30E−04 | 7.86E−05 (86%) | 1.60E−10 |
| | | | 1.50E+06 (30%) | | 7.06E−03 (14%) | |
| Parent 1 | H19/AS30 | 6.31E+05 | — | 5.23E−03 | | 8.29E−09 |
| Parent 2 | E3.2/AS30 | 5.80E+05 | 3.34E+05 (60%) | 1.72E−04 | 7.22E−05 (92%) | 1.25E−10 |
| | | | 1.22E+06 (40%) | | 1.58E−03 (8%) | |
| Biparatopic | H19/E3.2 | 5.74E+05 | 1.06E+06 (61%) | 1.71E−04 | 1.30E−04 (85%) | 2.26E−10 |
| | | | 1.41E+05 (39%) | | 9.98E−03 (15%) | |
| Parent 1 | TB19/AS30 | 1.65E+05 | — | 6.90E−04 | | 4.18E−09 |
| Parent 2 | TC29/AS30 | 7.59E+05 | — | 1.11E−04 | | 1.47E−10 |
| Biparatopic | TB19/TC29 | 6.85E+05 | 1.19E+06 (55%) | 8.96E−05 | 7.80E−05 (91%) | 1.14E−10 |
| | | | 1.16E+05 (45%) | | 6.00E−03 (9%) | |
| Parent 1 | TB19/AS30 | 1.65E+05 | — | 6.90E−04 | | 4.18E−09 |
| Parent 2 | H7/AS30 | 6.89E+05 | — | 1.95E−04 | | 2.83E−10 |
| Biparatopic | TB19/H7 | 7.52E+05 | 7.03E+05 (55%) | 1.49E−04 | 2.46E−04 (100%) | 3.28E−10 |
| | | | 5.31E+06 (45%) | | | |
| Parent 1 | F1.2/AS30 | 9.26E+05 | — | 3.12E−04 | | 3.36E−10 |
| Parent 2 | E3.2/AS30 | 5.80E+05 | 3.34E+05 (60%) | 1.72E−04 | 7.22E−05 (92%) | 1.25E−10 |
| | | | 1.22E+06 (40%) | | 1.58E−03 (8%) | |
| Biparatopic | F1.2/E3.2 | 9.39E+05 | 1.60E+06 (60%) | 1.24E−04 | 9.89E−05 (97%) | 1.05E−10 |
| | | | 4.09E+05 (40%) | | 8.59E−04 (3%) | |
| Parent 1 | H19/AS30 | 6.31E+05 | — | 5.23E−03 | | 8.29E−09 |
| Parent 2 | C16/AS30 | 5.74E+05 | 3.54E+05 (57%) | 3.93E−04 | 1.71E−04 (61%) | 2.99E−10 |
| | | | 1.26E+06 (44%) | | 8.66E−03 (38%) | |
| Biparatopic | H19/C16 | 4.54E+05 | 3.78E+05 (72%) | 3.66E−04 | 1.23E−04 (69%) | 2.70E−10 |
| | | | 1.31E+06 (28%) | | 3.48E−03 (31%) | |
| Parent 1 | CL25/AS30 | 5.54E+05 | — | 3.06E−03 | | 5.52E−09 |
| Parent 2 | TA10/AS30 | 3.40E+05 | — | 1.34E−04 | | 3.92E−10 |
| Biparatopic | CL25/TA10 | 5.70E+05 | 8.44E+05 (65%) | 2.20E−04 | 6.14E−05 (92%) | 1.08E−10 |
| | | | 1.88E+05 (35%) | | 5.36E−03 (7%) | |
| Parent 1 | TA9/AS30 | 5.21E+05 | 1.34E+06 (51%) | 5.13E−04 | 2.70E−04 (73%) | 5.18E−10 |
| | | | 2.65E+05 (49%) | | 1.29E−02 (25%) | |
| Parent 2 | H7/AS30 | 6.89E+05 | — | 1.95E−04 | | 2.83E−10 |
| Biparatopic | TA9/H7 | 5.97E+05 | 2.75E+05 (59%) | 2.19E−04 | 1.80E−04 (91%) | 3.01E−10 |
| | | | 1.60E+06 (41%) | | 1.86E−03 (9%) | |

*Langmuir ka with major component kd

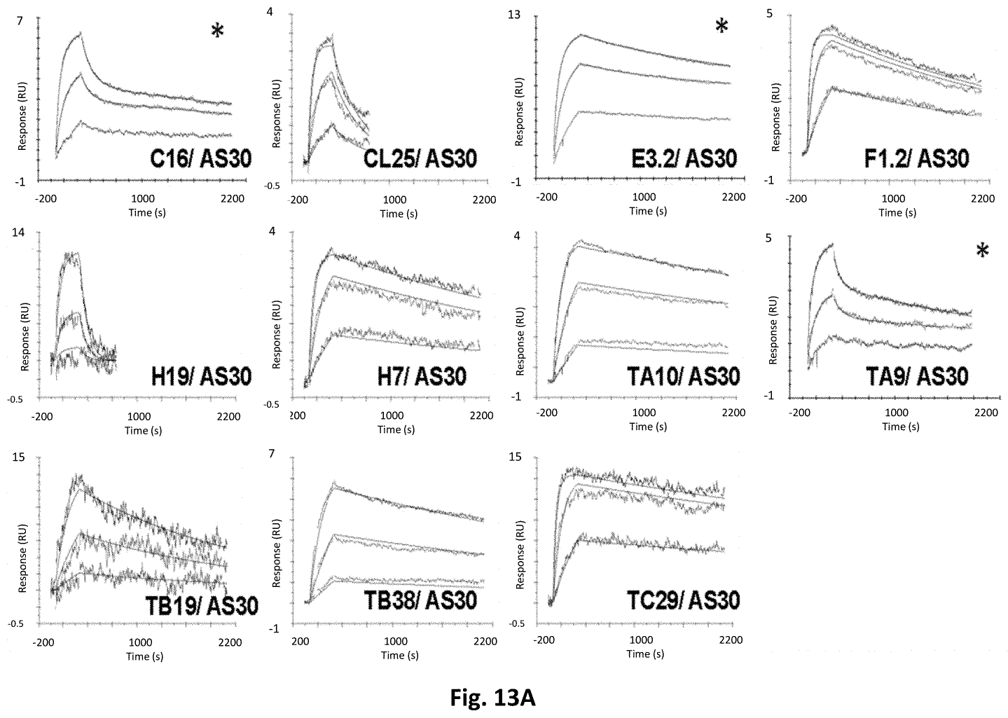

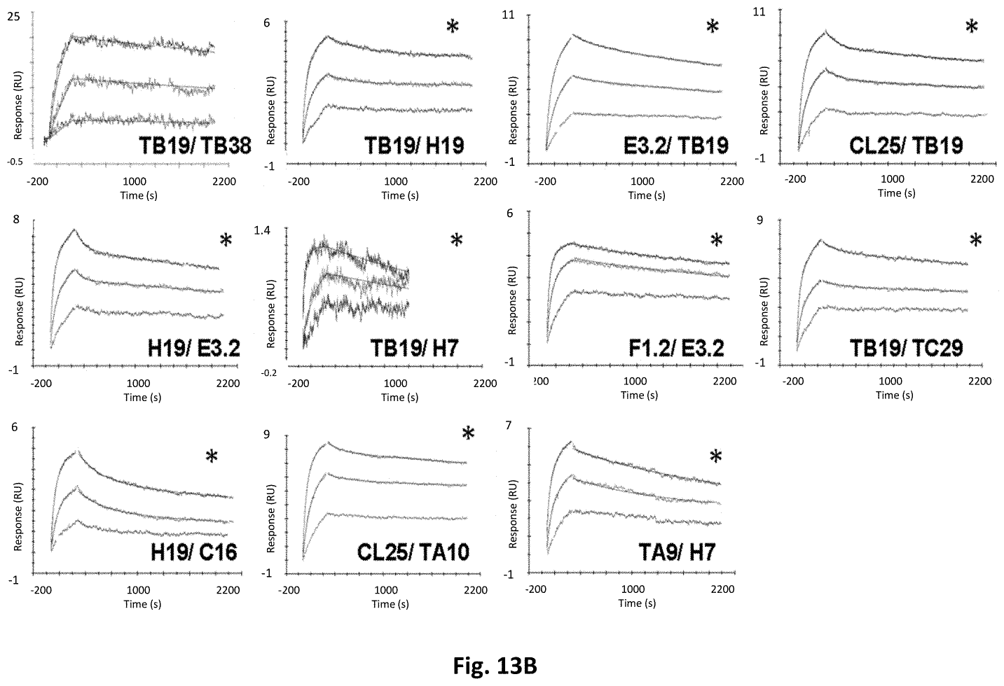

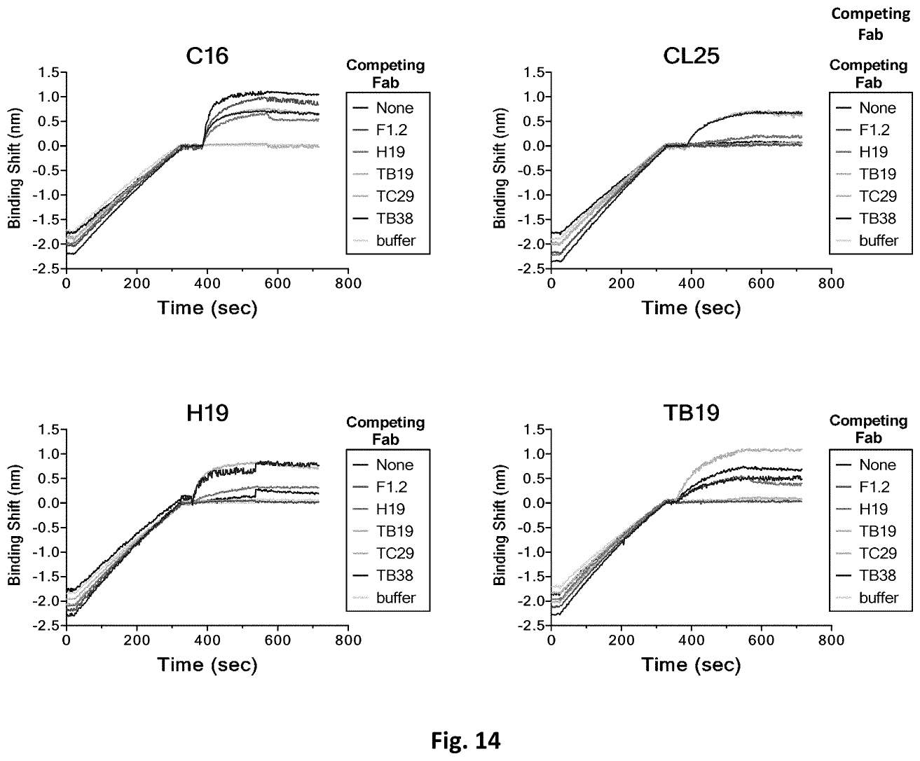

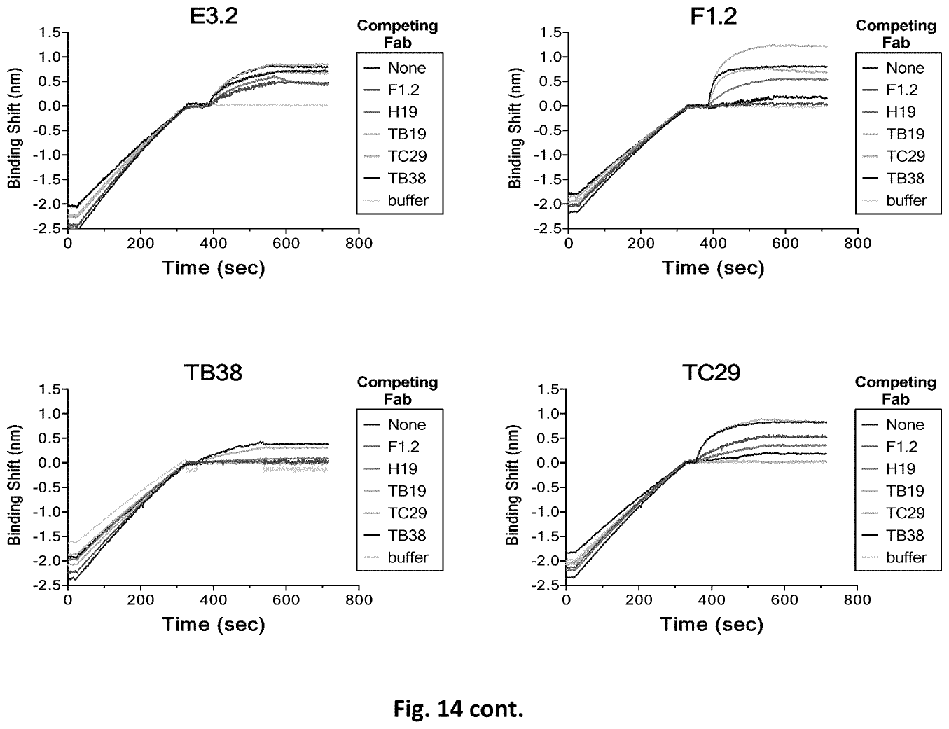

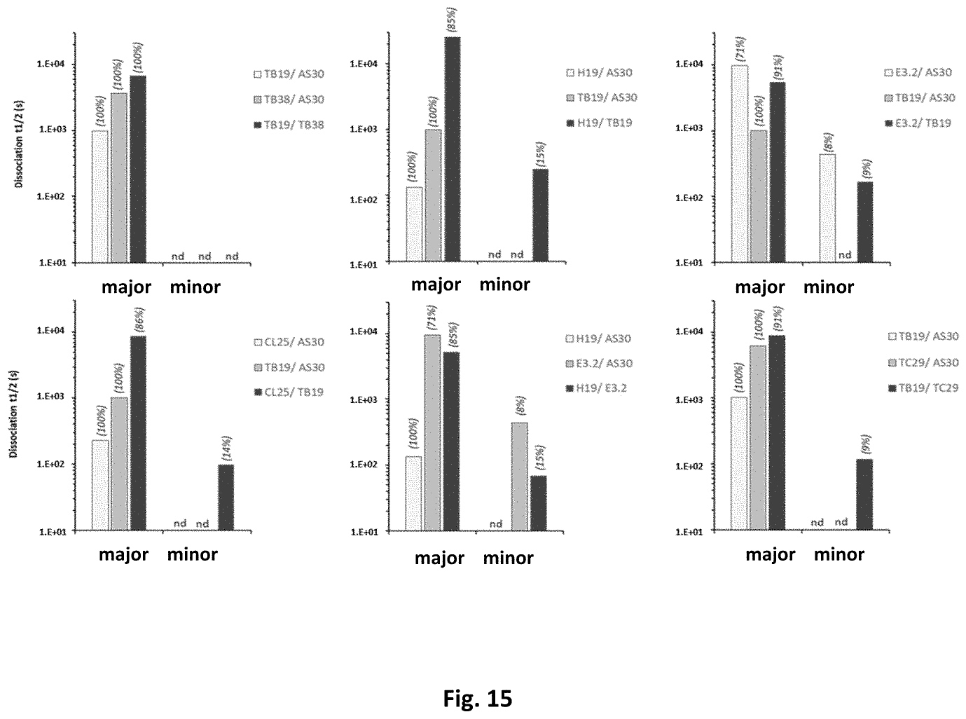

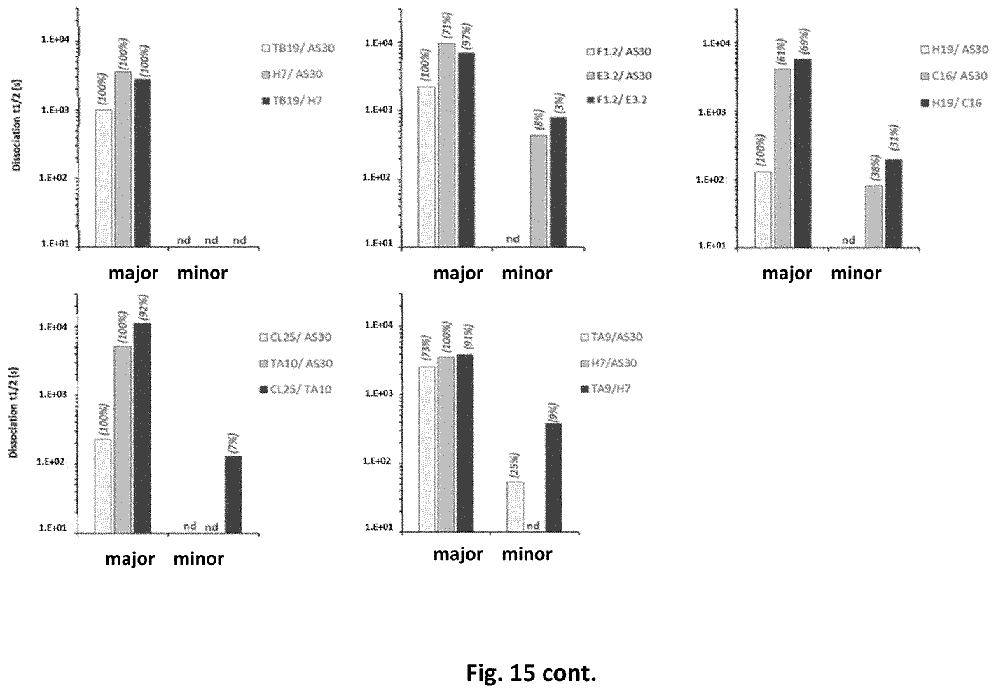

Nine out of eleven of the biparatopic antibodies displayed biphasic dissociation kinetics (FIG. 13A-13B) although largely as a consequence of a minor fraction (≤15%) of a faster-dissociating component. In one case (H19/C16) the abundance of this component was similar to that of the monovalent parent C16/AS30 (31% vs 38%), suggesting heterogeneity of the C16 monoclonal used for producing both. TA9/AS30 showed a similar heterogeneity (29% lower stability) which was not reflected in the biparatopic daughter TA9/H7. The half-times for dissociation compared with the monovalent parental antibodies are shown in FIG. 15. In eight of eleven cases the kd of the principal dissociation component was within 2.2-fold of the monovalent parent with the highest stability. In contrast, the difference between the kd values for the monovalent parental antibodies were on average 15-fold (range 1.5 to 73-fold, median 6.2) suggesting in these cases CD73 is bound by a single parental Fab arm on the immobilized antibody. However, in three cases (E3.2/TB19, CL25/TB19 and H19/TB19) the interaction with the biparatopic antibody was significantly more stable than with either monovalent parental (5.4, 8.8 and 26-fold respectively) suggesting the presence of additional contacts with the biparatopic antibody.

Bivalent kinetics of association were also apparent from a rapid increase in RU immediately following injection followed by a significant decline in rate after 100 seconds. Projection of the expected RU at early times from the kinetics after 100 seconds assuming pseudo first-order kinetics showed a significant residual consistent with a rapidly-binding component showing first order kinetics contributing a significant fraction of the RU after 300 seconds (30-49%). Fitting of both components by a reiterative process yielded a sum within ±0.2 of the observed RU over 90% of the course of binding (FIG. 13A-13B). Similar to the case of dissociation, the calculated ka values for each of the two components were within 3-fold of a monovalent parent (2.04±1.4 fold, range 1.02-2.71) in contrast to an average ˜6-fold difference between them (5.9±2.1, Table 6), suggesting they reflect independent binding to CD73 by each parental Fab arm.

Since each kinetic component for association cannot be unequivocally assigned to a specific one for dissociation, the relative affinities of the biparatopic antibodies and monovalent parental antibodies for CD73 were compared by KD values based on the kd value of the principal dissociation component and the ka value based on a Langmuir 1:1 binding model. The latter was within 30% of the average of the two ka components in the case of biphasic binding (Table 6). Consistent with the pattern seen for the kinetics of dissociation, the apparent affinity of the biparatopic variants (K_{D}) was similar to those of the more affine monovalent parental antibodies, indicating the interaction of the biparatopic antibodies could be largely attributed to binding of a single Fab arm. In two cases however (CL25/TB19 and TB19/H19) the biparatopic variant showed a significant increase over that of either monovalent parental antibody (26 and 69-fold respectively). This increase was specific to those combinations since the parents (TB19, H19, CL25) did not produce a similar enhancement with other partners. Since these increases required two cognate arms, it was inferred that this reflects the interaction of both arms of these biparatopic variants with CD73. In the majority of cases however, the affinity for CD73 was not increased by the addition of a second cognate Fab arm in spite of its being necessary for potency, suggesting interaction of the biparatopic antibody with an additional CD73 is required for potent inhibition on cells.

### Example 4—Epitope Binning

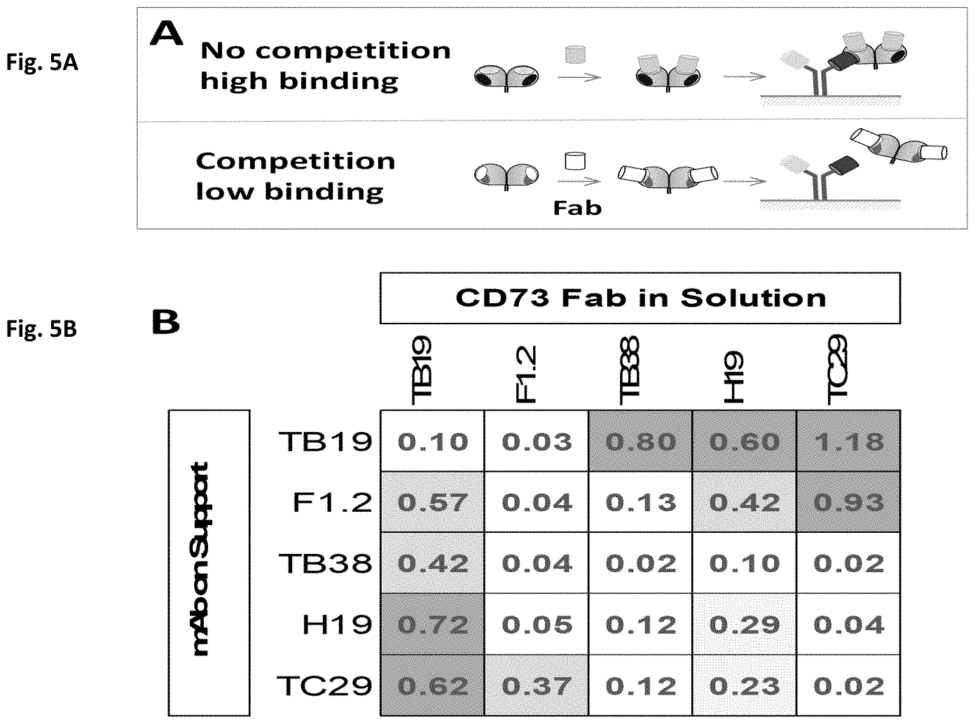

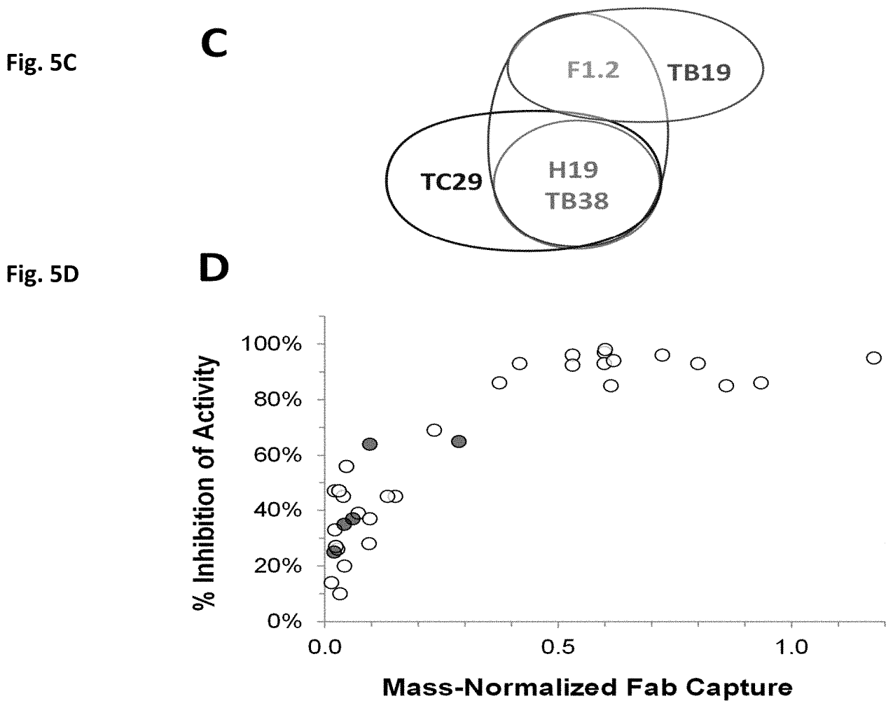

Epitopes of parental antibodies with the highest number of highly-potent combinations (TB19, E3.2, TB38, H19 and E3.2) were binned using biolayer interferometry (FIG. 5A). Monovalent IgG antibodies were used for coating on the solid support for the capture of CD73 from a mixture with competitor Fabs.

The result of interrogating a subset of the parental antibodies is shown in FIG. 5B. Higher values indicate capture of CD73 bound by the challenge Fab and no/low competition for binding (i.e., that the Fab binds to a CD73 epitope not overlapping that of the coated antibody) while lower values reflect blocking of the epitope by bound Fab for capture by the immobilized antibody. Allocation of the antibodies to different epitope bins based on these results is shown in FIG. 5C. One of the bins contained TB38, H19, and the mostly-overlapping TC29, all of which showed susceptibility to each of the Fabs except TB19. However, these three also showed differences in their susceptibilities to competition by different Fabs. For example, the capture of CD73 by a monovalent TB38 IgG was more susceptible to competition by H19 Fab than the capture either by TC29 or H19, while TC29 was distinguished from the other two by its partial resistance to competition by the F1.2 Fab, which was unique amongst all of the antibodies. While the bins were in most cases clearly delineated, intermediate levels of inhibition were observed in several cases (H19+H19, TC29+H19, TC29+F1.2, TB19+H19, F1.2+H19, and F1.2+TB19), possibly reflecting partially-overlapping epitopes (Abdiche et al. 2017. PloS one 12, e0169535) and/or significant differences in affinity. E3.2 could not be binned due to its aspecific interaction with the solid support.

Capture of a CD73::Fab complex by antibody in this binning experiment, reflecting a lack of competition between the parental antibodies, showed a high correlation with inhibition of cellular CD73 enzymatic activity by the corresponding biparatopic antibodies (FIG. 5D). Pairings of antibodies where more than 35% capture of a Fab was detected invariably produced ≥85% inhibition at 1 μg/mL as a biparatopic and, conversely, combinations with less than 35% capture achieved less than 70% inhibition as a biparatopic. These data indicate that to achieve high potency both antibodies comprising the biparatopic need to bind non-overlapping epitopes on CD73.

### Example 5— Structures of the TB19 and TB38 Fabs in Complex with CD73

Since the TB19 antibody successfully paired with a number of other antibodies including TB38 in the most potent biparatopic variants, it was important to understand the mechanism of action by examining their interactions with CD73 by structure analysis. Prior to preparing complexes with the TB19 and TB38 recombinant Fabs, the extracellular domain of human CD73 (residues 27-549) was deglycosylated with PNGaseF. The PNGaseF-treated product showed a molecular weight (MW) of 118 kDa by SEC-MALS, which was slightly larger than the polypeptide MW (116 kDa). This was attributable to a glycan observed in the structures at position Asn311, which was not susceptible to PNGase F cleavage. Crystallization parameters are shown in Table 7 below.

TABLE 7
Crystallographic parameters
Crystallographic data and refinement statistics

| | TB19 Fab:CD73 | | TB38 Fab:CD73 | |
|---|---|---|---|---|
| Diffraction data | | | | |
| Wavelength (Å) | 0.9762 | | 0.9763 | |
| Unit cell (Å) | 18.31, 74.22, 148.33 | | 236.91, 336.2, 222.15 | |
| Space group | [symbol] 1 2 1 | | C 2 2 2 | |
| Resolution range (Å) [symbol] | 59.04-2.25 | (2.33-2.25) | 78.61-3.73 | (3.863-3.73) |
| Data completeness (%) | 99.6 | (99.8) | 99.9 | (100) |
| Redundancy | 3.8 | (3.9) | 6.6 | (6.7) |
| Average I/σ(I) | 10.8 | (2.1) | 6 | (0.6) |
| R_{merge} | 0.071 | (0.693) | 0. [symbol] | (3.599) |

| | | |
|---|---|---|
| Refinement statistics | | |
| R_{work} (%) | 27.33 | 26.62 |
| R_{free} (%) | 31.62 | 31.48 |
| No. of atoms | | |
| Macromolecules | 5699 | 26275 |
| Ligands | 35 | 81 |
| B factors (average) | | |
| Macromolecules | 61.3 | 193.65 |
| Ligands | 72.56 | 207.51 |
| r.m.s.d. | | |
| Bond length (Å) | 0.009 | 0.003 |
| Bond angle (°) | 1.2 | 0.62 |
| Ramachandran plot (Å) | | |
| Favored | 91.57 | 94.07 |
| Allowed | 8.29 | 5.75 |

[symbol]

Values in parentheses are for the highest-resolution shell. [symbol]
indicates data missing or illegible when filed

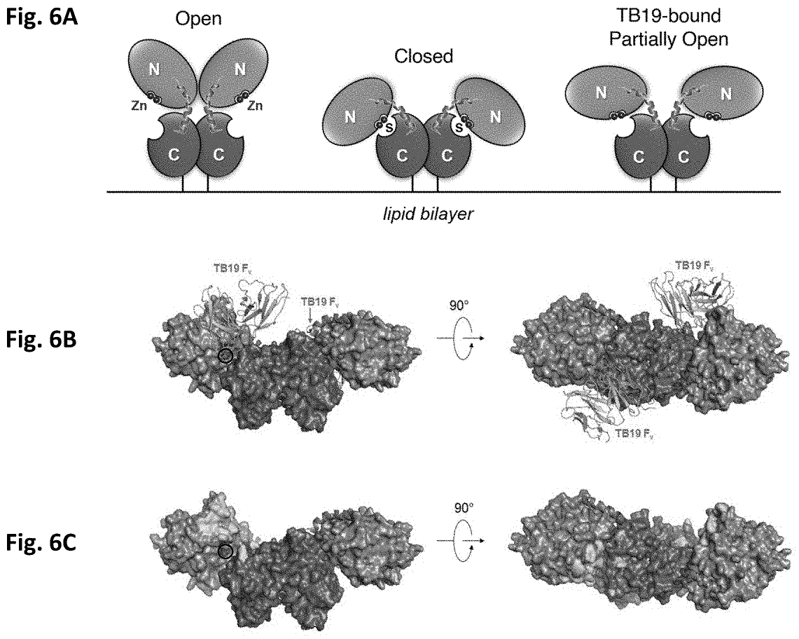

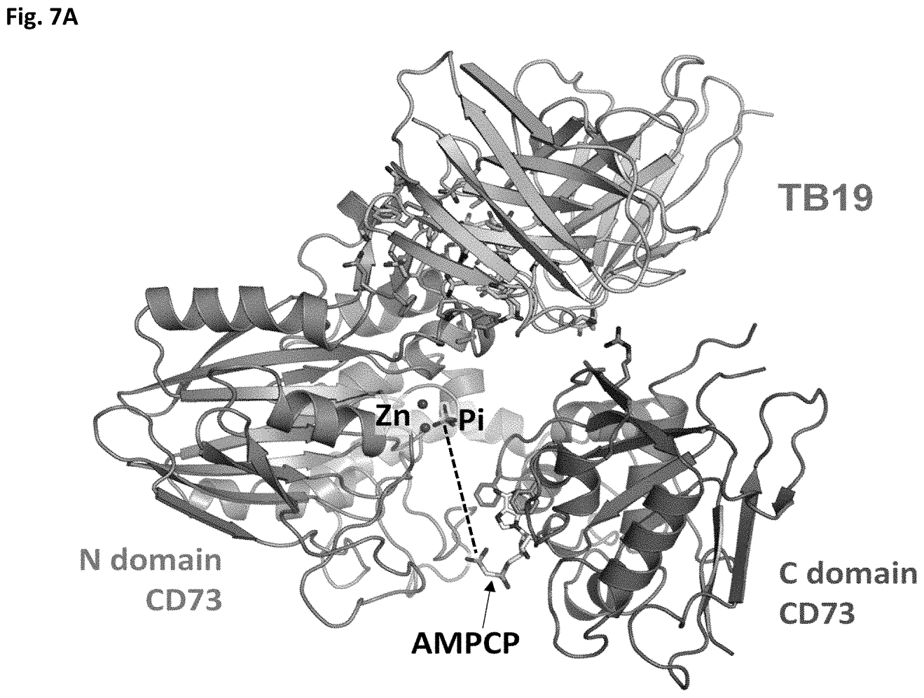

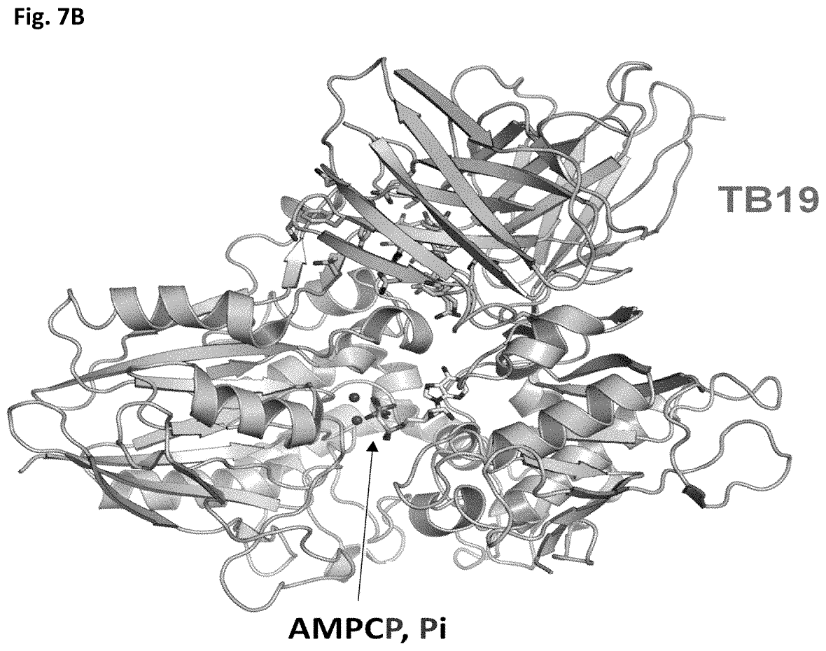

The structure of CD73 in complex with the TB19 Fab is shown in FIG. 6A-6B and FIG. 7A-7B. In the crystal asymmetric unit, one TB19 is bound to one CD73 monomer and only the Fv of the Fab could be built due to weak electron densities in the CH1/CL domains. A biological assembly of dimeric CD73 complex was obtained through a 2-fold crystallographic symmetry operation. In the resulting structure, CD73 dimerizes through an interface between the C-terminal domains (FIG. 6B), which closely resembles that of published structures (Heuts et al. 2012. Chembiochem: a European journal of chemical biology 13, 2384-2391; Knapp et al. 2012. Structure (London, England: 1993) 20, 2161-2173).

Within CD73 in the complex with TB19, well-defined positive densities are observed in the active site in the N-terminal domain. Two zinc ions and one phosphate were built accordingly and coordinated by residues Asp36, His38, Asp85, Asn117, His118, His220 and His243 in the catalytic center, as the TB19 complex was crystallized in the presence of phosphate. These zinc ions and phosphate are in the same position as the two zinc ions and the β-phosphonate of the substrate analog AMPCP in the closed conformer of CD73 (PDB 4H2I) (FIG. 7A-7B). The conserved dimerization interface and position of the zincs and phosphate indicate the structure of the CD73 dimer in the complex with TB19 is biologically relevant.

CD73 has been previously reported in either an open or a closed conformation, depending on the absence or presence of substrate in the active site, respectively (Knapp, supra) (FIG. 6A). However, when bound by TB19, CD73 takes on a conformation in which the N- and C-terminal domains are in an intermediate position between those previously reported for the open and closed conformers (FIG. 6A-6B and FIG. 7A-7B). When the C-terminal domains of earlier structures and TB19-bound CD73 are superimposed, the position of the zinc-coordinating residue H220 in the N-terminal domain is approximately 22 Å away from its position in the closed conformer (PDB 4H2I) and 27 Å away from that in the open conformer (PDB 4H2F).

All of the TB19 CDR loops except CDRL2 contact a portion of the N-terminal domain adjacent to the zinc and phosphate binding site (FIG. 7A-7B), although none of the antibody residues directly interact with any of the catalytic center forming residues. In addition, the TB19 CDRH2 residue Ser62 and CDRL1 residue Ser26 (FIG. 6B, FIG. 6C) are spatially close to the C-terminal domain, but 20 Å away from the substrate binding residues including Arg354, Asn390, Arg395, Phe417, Phe500, and Asp506. In the presence of TB19 those substrate-binding residues are far from the catalytic center and the zincs in the N-terminal domain. For example, the residues Phe417 and Phe500 which bind the adenine ring are 11-13 Å from their positions in the closed conformer with substrate (PDB 4H2I).

Because of the orientation of TB19 and its epitope location, clashes between C-terminal domain and TB19 are observed when superimposing the N-terminal domains of CD73 in our structure and the closed conformer of CD73 (FIG. 7A-7B). Thus, bound TB19 will block the alignment of N- and C-terminal domains in CD73 and prevent formation of the closed conformer. As a result, TB19 binding will separate the zinc ions and catalytic residues of the N-terminal domain from the phosphoanhydride bond of the substrate, thereby blocking enzymatic activity.

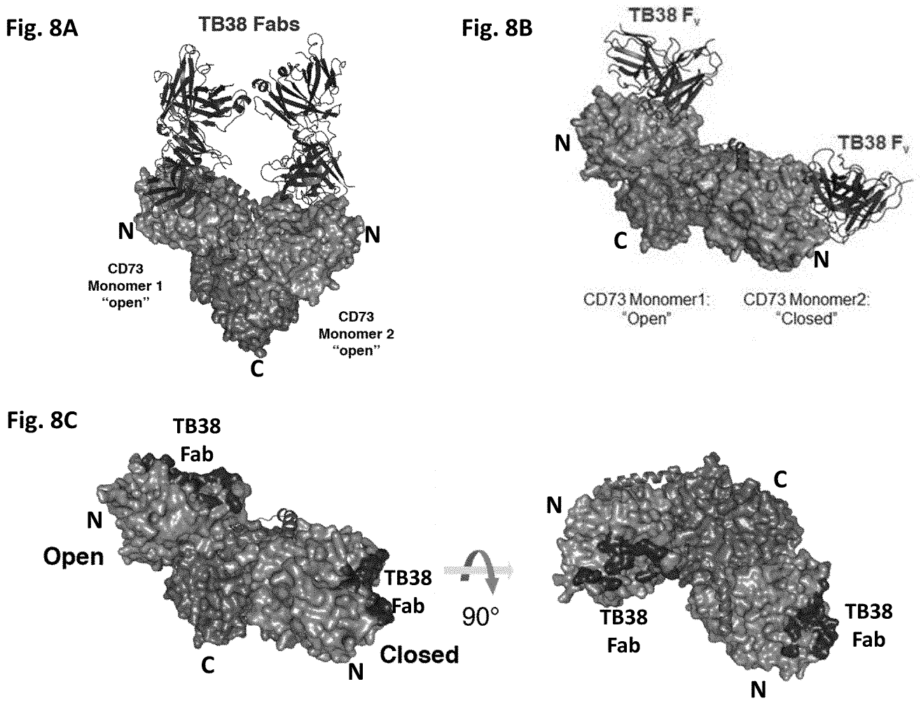

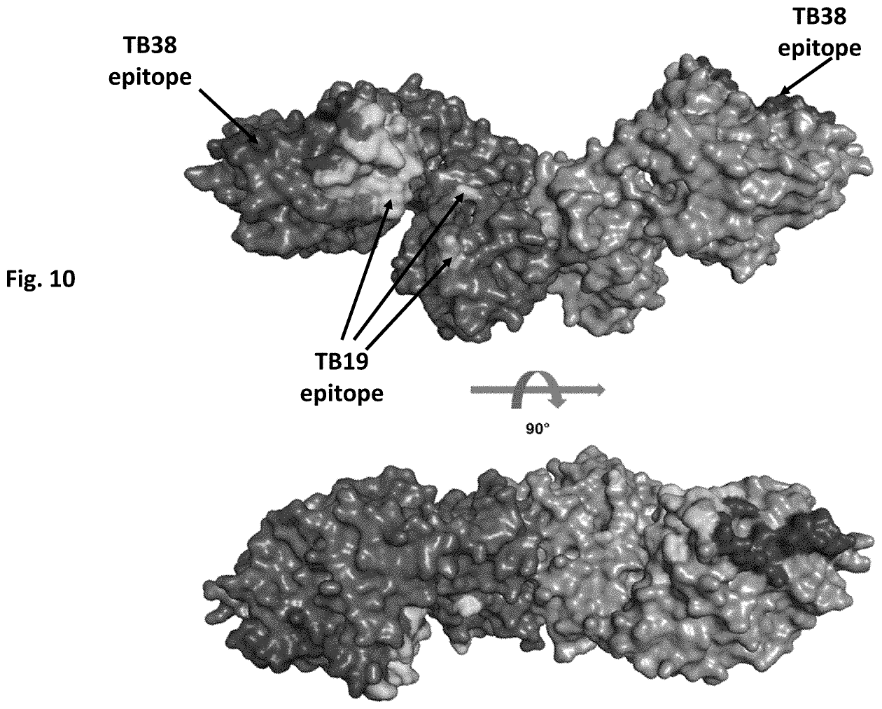

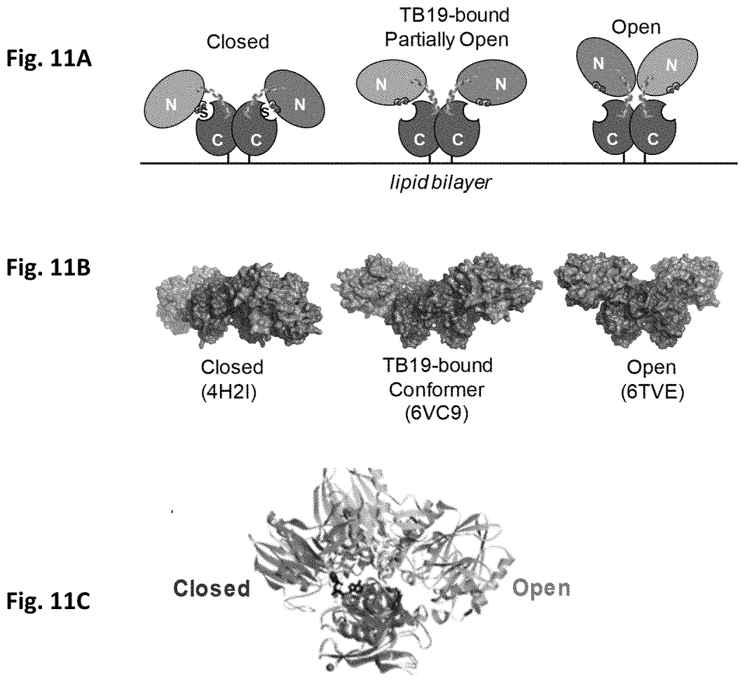

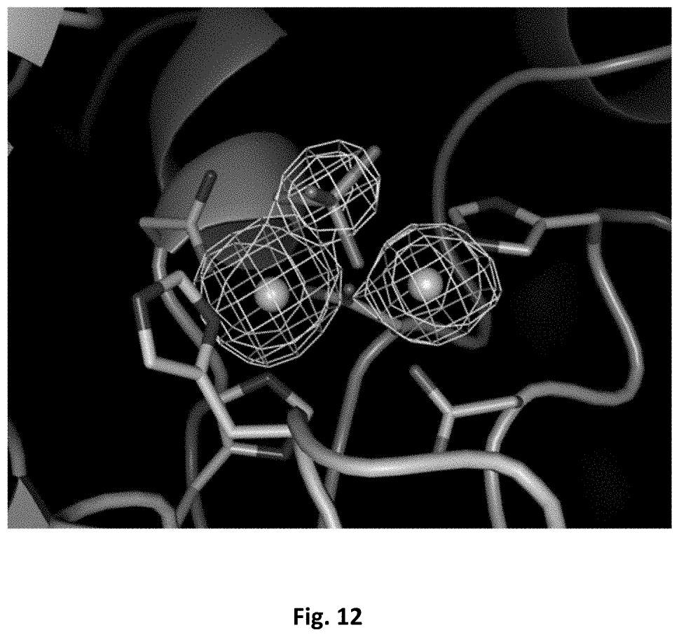

In contrast to TB19, the TB38 Fab and CD73 yielded structures with each asymmetric unit containing two CD73 dimers in different conformations with all of the monomers bound by one Fab (FIG. 8A-8C). In the first structure (FIG. 8A), electron densities for the CH1/CL domains were well-defined and the full Fab structure could be built. In the second (FIG. 8B), weak density for the constant domains was observed so only the Fv domains were built. Strikingly, the conformation of CD73 in the two structures is different. In the first, CD73 is in a symmetrical open conformation which can be superimposed on the canonical open conformer in PDB 4H2F with a root mean square deviation value of 1 Å. However, the CD73 dimer in the second structure is in a non-symmetrical conformation which has not been reported previously in which the monomers are in different conformations. In this hybrid structure, one monomer is in the open conformation previously observed in a crystal with bound adenosine (PDB 4H2F) while the other is in the closed conformation seen in the presence of the substrate analog AMPCP (PDB 4H2I) (Knapp, supra). In both complexes, the TB38 Fab contacts residues solely in the N-terminal domain (including Lys145, Ser152, Ser155, Gly156, Leu159-Lys162, Glu203, Lys206, Leu210, and Asn211) and all 6 CDRs are engaged in the interactions. Mapping of the epitope residues of TB19 and TB38 on the partially-open structure of CD73 (FIG. 10) and by sequence alignment show that the epitopes are non-overlapping, albeit in close proximity in agreement with the binning results.

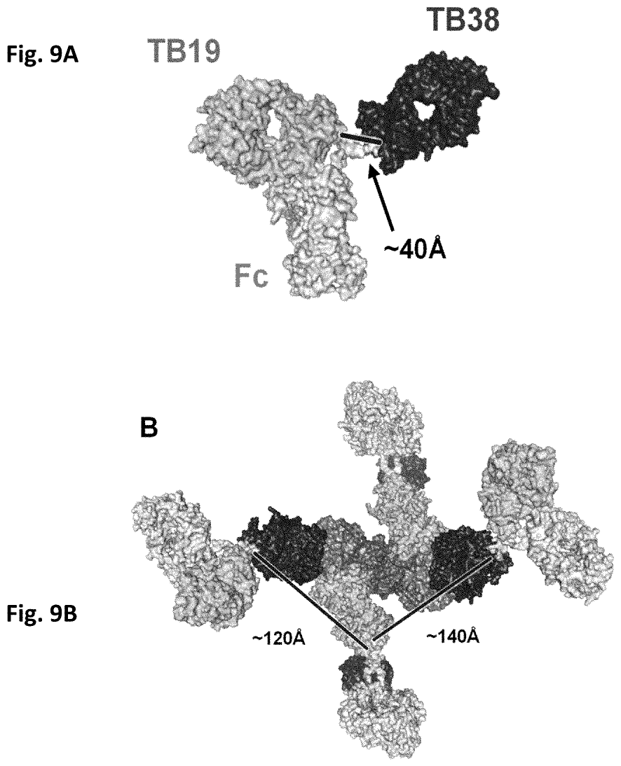

To assess possible engagement of CD73 dimer by a bispecific TB19/TB38 antibody, an IgG was modelled by replacing the Fv's of a complete IgG antibody structure (PDB 1HZH) with those of TB19 and TB38 (FIG. 9A-9B). The distance between the CH1 domains of TB19 and TB38 in this model (FIG. 9A) is approximately 40 Å (measured between the Ca of Ala225 of the CH1 domain). Modeling bivalent binding to CD73 in the partly-open conformation by this biparatopic was not possible, either by binding the two epitopes on the same or opposing monomers, although each CD73 monomer could be bound by two antibodies monovalently as illustrated in FIG. 9B. In order for a single antibody to bind the CD73 dimer bivalently, the C-terminal residue of the Fab CH1 domains would need to be separated by ˜120 Å and ˜140 Å to bind the epitopes either on the same or opposite monomers respectively, which is much further than can be achieved by an IgG. It was concluded that it is likely that a biparatopic TB19/TB38 antibody would be incapable of binding a single CD73 dimer in a bivalent manner.

What is claimed:

1. An antigen-binding protein or fragment thereof with binding specificity to a CD73 epitope, comprising:
(a) an antibody heavy chain variable (VH) domain comprising a CDR-H1 sequence comprising the amino acid sequence of GGSIRNNY (SEQ ID NO: 1) or GFTFSSYG (SEQ ID NO: 7), a CDR-H2 sequence comprising the amino acid sequence of IYISGTT (SEQ ID NO: 2) or FWYDGSNK (SEQ ID NO: 8), and a CDR-H3 sequence comprising the amino acid sequence of AREHYVSGTSLDN (SEQ ID NO: 3) or ARAPNWDDAFDI (SEQ ID NO: 9); and
(b) an antibody light chain variable (VL) domain comprising a CDR-L1 sequence comprising the amino acid sequence of QSVNTNY (SEQ ID NO: 4) or SGSVSTSYY (SEQ ID NO: 10), a CDR-L2 sequence comprising the amino acid sequence of GTS (SEQ ID NO: 5) or STN (SEQ ID NO: 11), and a CDR-L3 sequence comprising the amino acid sequence of QQDYNLPYT (SEQ ID NO: 6) or VLFMGSGIWV (SEQ ID NO: 12).

2. The antigen binding protein or fragment thereof of claim 1, wherein the VH domain comprises the amino acid sequence of SEQ ID NO: 13 or SEQ ID NO: 15, and the VL domain comprises the amino acid sequence of SEQ ID NO: 14 or SEQ ID NO: 16.

3. The antigen binding protein or fragment thereof of claim 1, wherein the antibody heavy chain comprises the amino acid sequence of SEQ ID NO: 17, 18, 20, or 21, and the antibody light chain comprises the amino acid sequence of SEQ ID NO: 19 or SEQ ID NO: 22.

4. The antigen binding protein or fragment thereof of claim 2, comprising a VH domain at least about 90% identical or at least about 95% identical to the amino acid sequence of SEQ ID NO: 13 or SEQ ID NO: 15, and a VL domain at least about 90% identical or at least about 95% identical to the amino acid sequence of SEQ ID NO: 14 or SEQ ID NO: 16.

5. The antigen binding protein or fragment thereof of claim 3, comprising an antibody heavy chain at least about 90% identical or at least about 95% identical to the amino acid sequence of SEQ ID NO: 17, 18, 20, or 21, and an antibody light chain at least about 90% identical or at least about 95% identical to the amino acid sequence of SEQ ID NO: 19 or SEQ ID NO: 22.

6. The antigen binding protein or fragment thereof of claims 1-5, wherein:
(a) the VH domain comprises a CDR-H1 sequence comprising the amino acid sequence of GGSIRNNY (SEQ ID NO: 1), a CDR-H2 sequence comprising the amino acid sequence of IYISGTT (SEQ ID NO: 2), and a CDR-H3 sequence comprising the amino acid sequence of AREHYVSGTSLDN (SEQ ID NO: 3); and
(b) the VL domain comprises a CDR-L1 sequence comprising the amino acid sequence of QSVNTNY (SEQ ID NO: 4), a CDR-L2 sequence comprising the amino acid sequence of GTS (SEQ ID NO: 5), and a CDR-L3 sequence comprising the amino acid sequence of QQDYNLPYT (SEQ ID NO: 6).

7. The antigen binding protein or fragment thereof of claim 6, wherein the VH domain comprises the amino acid sequence of SEQ ID NO: 13, and the VL domain comprises the amino acid sequence of SEQ ID NO: 14.

8. The antigen binding protein or fragment thereof of claim 6, wherein the antibody heavy chain comprises the amino acid sequence of SEQ ID NO: 17 or SEQ ID NO: 18, and the antibody light chain comprises the amino acid sequence of SEQ ID NO: 19.

9. The antigen binding protein or fragment thereof of claims 1-5, wherein:
(a) the VH domain comprises a CDR-H1 sequence comprising the amino acid sequence of GFTFSSYG (SEQ ID NO: 7), a CDR-H2 sequence comprising the amino acid sequence of FWYDGSNK (SEQ ID NO: 8), and a CDR-H3 sequence comprising the amino acid sequence of ARAPNWDDAFDI (SEQ ID NO: 9); and
(b) the VL domain comprises a CDR-L1 sequence comprising the amino acid sequence of SGSVSTSYY (SEQ ID NO: 10), a CDR-L2 sequence comprising the amino acid sequence of STN (SEQ ID NO: 11), and a CDR-L3 sequence comprising the amino acid sequence of VLFMGSGIWV (SEQ ID NO: 12).

10. The antigen binding protein or fragment thereof of claim 9, wherein the VH domain comprises the amino acid sequence of SEQ ID NO: 15, and the VL domain comprises the amino acid sequence of SEQ ID NO: 16.

11. The antigen binding protein or fragment thereof of claim 9, wherein the antibody heavy chain comprises the amino acid sequence of SEQ ID NO: 20 or SEQ ID NO: 21, and the antibody light chain comprises the amino acid sequence of SEQ ID NO: 22.

12. The antigen binding protein or fragment thereof of claims 1-11, wherein the antigen binding protein binds a human CD73 polypeptide comprising the amino acid sequence of SEQ ID NO: 23.

13. The antigen binding protein or fragment thereof of claims 1-8 and 12, wherein the antigen binding protein binds an epitope of human CD73 polypeptide comprising the amino acids N96, G97, V98, E99, K121, P123, P156, F157, S159, N160, G162, T163, N164, L165, V166, F167, E168, R491, and D496 of SEQ ID NO: 23.

14. The antigen binding protein or fragment thereof of claims 1-5 and 9-12, wherein the antigen binding protein binds an epitope of human CD73 polypeptide comprising the amino acids P112, K119, A125, S126, S129, G130, L133, P134, Y135, K136, K180, L184, and N185 of SEQ ID NO: 23.

15. The antigen binding protein or fragment thereof of claims 1-14 wherein the antigen binding protein is a chimeric or humanized antibody.

16. The antigen binding protein or fragment thereof of claims 1-14, wherein the antigen binding protein is a human antibody.

17. The antigen binding protein or fragment thereof of claims 1-16, wherein the antigen binding protein is a monoclonal antibody.

18. The antigen binding protein or fragment thereof of claims 1-17, wherein the antigen binding protein comprises one or more full-length antibody heavy chains comprising an Fc region.

19. The antigen binding protein or fragment thereof of claim 18, wherein the Fc region is a human IgG1 Fc region.

20. The antigen binding protein or fragment thereof of claim 19, wherein the human IgG1 Fc region comprises amino acid substitutions at one or more positions corresponding to positions 405 and 409 of human IgG1 according to EU Index, wherein the amino acid substitutions are F405L and K409R.

21. A pharmaceutical composition comprising the antigen binding protein or fragment thereof of any one of the preceding claims, and a pharmaceutically acceptable carrier.

22. An isolated nucleic acid molecule encoding the antigen binding protein or fragment thereof of any one of the preceding claims.

23. An expression vector comprising the nucleic acid molecule of claim 22.

24. A host cell comprising the expression vector of claim 23.

25. A biparatopic antigen-binding protein comprising binding specificity to a first CD73 epitope and a second CD73 epitope.

26. The biparatopic antigen-binding protein of claim 25, comprising:
(a) a first VH domain with specificity to the first CD73 epitope comprising a CDR-H1 sequence comprising the amino acid sequence of GGSIRNNY (SEQ ID NO: 1), a CDR-H2 sequence comprising the amino acid sequence of IYISGTT (SEQ ID NO: 2), and a CDR-H3 sequence comprising the amino acid sequence of AREHYVSGTSLDN (SEQ ID NO: 3);
(b) a first VL domain with specificity to the first CD73 epitope comprising a CDR-L1 sequence comprising the amino acid sequence of QSVNTNY (SEQ ID NO: 4), a CDR-L2 sequence comprising the amino acid sequence of GTS (SEQ ID NO: 5), and a CDR-L3 sequence comprising the amino acid sequence of QQDYNLPYT (SEQ ID NO: 6);
(c) a second VH domain with specificity to the second CD73 epitope comprises a CDR-H1 sequence comprising the amino acid sequence of GFTFSSYG (SEQ ID NO: 7), a CDR-H2 sequence comprising the amino acid sequence of FWYDGSNK (SEQ ID NO: 8, and a CDR-H3 sequence comprising the amino acid sequence of ARAPNWDDAFDI (SEQ ID NO: 9); and
(d) a second VL domain with specificity to the second CD73 epitope comprises a CDR-L1 sequence comprising the amino acid sequence of SGSVSTSYY (SEQ ID NO: 10), a CDR-L2 sequence comprising the amino acid sequence of STN (SEQ ID NO: 11), and a CDR-L3 sequence comprising the amino acid sequence of VLFMGSGIWV (SEQ ID NO: 12.

27. The biparatopic antigen-binding protein of claim 25, wherein:
the first VH domain comprises the amino acid sequence of SEQ ID NO: 13;
the second VH domain comprises the amino acid sequence of SEQ ID NO: 15;
the first VL domain comprises the amino acid sequence of SEQ ID NO: 14; and
the second VL domain comprises the amino acid sequence of SEQ ID NO: 16.

28. The biparatopic antigen-binding protein of claim 25, wherein:
(a) a first antibody heavy chain comprises the amino acid sequence of SEQ ID NO: 17 or SEQ ID NO: 18;
(b) a second antibody heavy chain comprises the amino acid sequence of SEQ ID NO: 20 or SEQ ID NO: 21;
(c) a first antibody light chain comprises the amino acid sequence of SEQ ID NO: 19; and
(d) a second antibody light chain comprises the amino acid sequence of SEQ ID NO: 22.

29. The biparatopic antigen-binding protein of claim 28, wherein:
(a) a first antibody heavy chain comprises the amino acid sequence of SEQ ID NO: 17;
(b) a second antibody heavy chain comprises the amino acid sequence of SEQ ID NO: 21;
(c) a first antibody light chain comprises the amino acid sequence of SEQ ID NO: 19; and
(d) a second antibody light chain comprises the amino acid sequence of SEQ ID NO: 22.

30. The biparatopic antigen-binding protein of claim 28, wherein:
(a) a first antibody heavy chain comprises the amino acid sequence of SEQ ID NO: 18;
(b) a second antibody heavy chain comprises the amino acid sequence of SEQ ID NO: 20;
(c) a first antibody light chain comprises the amino acid sequence of SEQ ID NO: 19; and
(d) a second antibody light chain comprises the amino acid sequence of SEQ ID NO: 22.

31. The biparatopic antigen-binding protein of claims 25-30, wherein:
(a) the first VH and VL domains bind a first epitope of human CD73 polypeptide comprising the amino acids N96, G97, V98, E99, K121, P123, P156, F157, S159, N160, G162, T163, N164, L165, V166, F167, E168, R491, and D496 of SEQ ID NO: 23; and
(b) the second VH and VL domains bind a second epitope of human CD73 polypeptide comprising the amino acids P112, K119, A125, S126, S129, G130, L133, P134, Y135, K136, K180, L184, and N185 of SEQ ID NO: 23.

32. The biparatopic antigen-binding protein of claims 25-31, comprising higher inhibitory activity of CD73 compared to one or both of the monospecific parental antibodies.

33. The biparatopic antigen-binding protein of claims 25-31, comprising higher inhibitory activity of CD73 compared to the combination of monospecific parental antibodies.

34. The biparatopic antigen-binding protein of claims 25-33, wherein the first VH and VL domains bind a first CD73 epitope on a first CD73 dimer molecule, and the second VH and VL domains bind a second CD73 epitope on a second CD73 dimer molecule.

35. The biparatopic antigen-binding protein of claims 25-33, wherein the antigen-binding protein is capable of crosslinking two or more CD73 dimer molecules.

36. The biparatopic antigen-binding protein of claim 25, produced by Fab arm exchange.

37. The biparatopic antigen-binding protein of claim 36, wherein the Fab arm exchange is performed following the steps of:
(a) mixing a first parental, monospecific antigen-binding protein comprising an IgG1 Fc region comprising an amino acid substitution F405L according to EU Index, and a second parental, monospecific antigen-binding protein comprising an IgG1 Fc region comprising an amino acid substitution K409R according to EU Index, to produce a mixture;
(b) placing the mixture of step (a) under reducing conditions to produce a reduced antigen-binding protein mixture containing the biparatopic, bispecific antigen-binding protein;
(c) placing the mixture of step (b) under oxidizing conditions to reform the disulfide linkages between the heavy chains of the biparatopic, bispecific antigen-binding protein; and
(d) isolating the biparatopic, bispecific antigen-binding protein.

38. The biparatopic antigen-binding protein of claim 36, wherein the first parental, monospecific antigen-binding protein and second parental, monospecific antigen-binding protein are mixed at equimolar amounts.

39. The biparatopic antigen-binding protein of claim 36, wherein the reducing conditions are produced by adding a reducing agent.

40. The biparatopic antigen-binding protein of claim 39, wherein the reducing agent comprises mercaptoethylamine (MEA).

41. The biparatopic antigen-binding protein of claim 36, wherein the mixture of step (a) is placed under reducing conditions for about 3 hours to about 6 hours at a temperature of about 18° C. to about 30° C.

42. A method for treating a CD73-mediated disease or disorder in a subject, comprising administering to a subject in need thereof the antigen binding protein or fragment thereof of any one of the preceding claims.

43. The method of claim 42, wherein the CD73-mediated disease or disorder is cancer.

44. A method of selecting biparatopic antigen-binding proteins comprising higher inhibitory activity of CD73 compared to one or more monospecific parental antibodies, the method comprising the steps of:
a) combining two parental antibodies under conditions that form a biparatopic antigen-binding protein;
b) testing the biparatopic antigen-binding protein and one or both of the two parental antibodies in a CD73 activity assay;
c) comparing the CD73 activity with the biparatopic antigen-binding protein to the CD73 activity with one or both of the two parental antibodies; and
d) selecting the biparatopic antigen-binding protein if the CD73 activity is less than the CD73 activity of one or both of the two parental antibodies.

45. The method of claim 44, wherein the CD73 activity assay measures adenosine formation.

46. The method of claim 45, wherein the adenosine formation is quantitated by liquid chromatography-mass spectrometry (LC/MS).

47. The method of claim 44, wherein the CD73 activity assay is performed with COR-L23 lung carcinoma cells expressing human CD73.
